SEQUENCE LISTING (31 sequences)

SEQ ID NO: 1 - protein, 20 aa, Artificial Sequence
  AG20
ALGEQKLISEEDLGLPETGG

SEQ ID NO: 2 - protein, 324 aa, Artificial Sequence
  Peptide
ARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGFEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQ

SEQ ID NO: 3 - protein, 328 aa, Artificial Sequence
  Peptide
ARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGFEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDAD

SEQ ID NO: 4 - protein, 5 aa, Artificial Sequence
  Peptide linker
EAAAK

SEQ ID NO: 5 - protein, 15 aa, Artificial Sequence
  Peptide linker
EAAAKEAAAKEAAAK

SEQ ID NO: 6 - protein, 25 aa, Artificial Sequence
  Peptide linker
EAAAKEAAAKEAAAKEAAAKEAAAK

SEQ ID NO: 7 - protein, 21 aa, Artificial Sequence
  B-EK15-LPETGG
EAAAKEAAAKEAAAKLPETGG

SEQ ID NO: 8 - protein, 22 aa, Artificial Sequence
  B-EK15-LPETGGG
EAAAKEAAAKEAAAKLPETGGG

SEQ ID NO: 9 - protein, 18 aa, Artificial Sequence
  B-EK15-NDL
EAAAKEAAAKEAAAKNDL

SEQ ID NO: 10 - protein, 18 aa, Artificial Sequence
  EK18
EAAAKEAAAKEAAAKNGL

SEQ ID NO: 11 - protein, 18 aa, Artificial Sequence
  Peptide linker
EAAAKEAAAKEAAAKNPL

SEQ ID NO: 12 - protein, 10 aa, Artificial Sequence
  Peptide linker
EQKLISEEDL

SEQ ID NO: 13 - protein, 20 aa, Artificial Sequence
  GG20
GGGEQKLISEEDLGLPETGG

SEQ ID NO: 14 - protein, 17 aa, Artificial Sequence
  EL17
GGGEQKLISEEDLGNGL

SEQ ID NO: 15 - protein, 21 aa, Artificial Sequence
  GA20
GLAEQKLISEEDLGGLPETGG

SEQ ID NO: 16 - protein, 34 aa, Artificial Sequence
  GG39
GLGEAAAKEAAAKEAAAKEAAAKEAAAKLPETGG

SEQ ID NO: 17 - protein, 20 aa, Artificial Sequence
  GN20
GLGEQKLISEEDLGLPETGG

SEQ ID NO: 18 - protein, 17 aa, Artificial Sequence
  GL17
GLGEQKLISEEDLGNGL

SEQ ID NO: 19 - protein, 25 aa, Artificial Sequence
  GK25
GLGSSGSGGDYKDDDDKGGSGSGGK

SEQ ID NO: 20 - protein, 8 aa, Artificial Sequence
  B-GG8
GLPETGGG

SEQ ID NO: 21 - protein, 20 aa, Artificial Sequence
  TL20
YHWYGYTPQNVIGGGGSNGL

SEQ ID NO: 22 - protein, 29 aa, Artificial Sequence
  GL29
GSSGSGGEQKLISEEDLGGSGGSGSGNGL

SEQ ID NO: 23 - protein, 147 aa, Artificial Sequence
  Peptide linker
HHHHHHGSGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGSGDYKDDDDKNGL

SEQ ID NO: 24 - protein, 7 aa, Artificial Sequence
  Peptide linker
LGETGGG

SEQ ID NO: 25 - protein, 6 aa, Artificial Sequence
  Peptide linker
LPETGG

SEQ ID NO: 26 - protein, 7 aa, Artificial Sequence
  Peptide linker
LPETGGG

SEQ ID NO: 27 - protein, 5 aa, Artificial Sequence
  Peptide linker
  misc_feature (3)..(3): Xaa can be any naturally occurring amino acid
LPXTG

SEQ ID NO: 28 - protein, 6 aa, Artificial Sequence
  Peptide linker
  misc_feature (3)..(3): Xaa can be any naturally occurring amino acid
LPXTGG

SEQ ID NO: 29 - protein, 5 aa, Artificial Sequence
  Peptide linker
NSLAN

SEQ ID NO: 30 - protein, 4 aa, Artificial Sequence
  Peptide linker
NSLD

SEQ ID NO: 31 - protein, 5 aa, Artificial Sequence
  TL5
TRNGL

(19) United States
(12) Patent Application Publication
(10) Pub. No.: US 2024/0110153 A1
(43) Pub. Date: Apr. 4, 2024

(54) Surface Modified Red Blood Cells And Methods Of Generating The Same

(71) Applicants: NATIONAL UNIVERSITY OF SINGAPORE, Singapore (SG); CITY UNIVERSITY OF HONG KONG, Hong Kong (CN)

(72) Inventors: Thi Nguyet Minh LE, Singapore (SG); Migara Kavishka JAYASINGHE, Singapore (SG); Boya PENG, Singapore (SG); Jiahai SHI, Hong Kong (CN)

(73) Assignees: NATIONAL UNIVERSITY OF SINGAPORE, Singapore (SG); CITY UNIVERSITY OF HONG KONG, Hong Kong (CN)

(21) Appl. No.: 18/274,649

(22) PCT Filed: Jan. 28, 2022
(86) PCT No.: PCT/SG2022/050047

§ 371 Date: Jul. 27, 2023

(30) Foreign Application Priority Data
Jan. 29, 2021 (SG) 10202101003S

(51) Int. Cl. C12N 5/078; C12N 9/00

(52) U.S. Cl. CPC C12N 5/0641; C12N 9/93; C12Y 601/01022

(57) ABSTRACT

The present invention relates to methods modifying cell surface markers of red blood cells (RBCs) and uses of the same. In particular, the method comprises contacting an RBC with a peptide in the presence of a ligase, under suitable conditions and for sufficient time to allow ligation of the peptide to the RBC to form an RBC-peptide conjugate. In one embodiment, the ligase is OaAEPI ligase. The RBC-peptide conjugate may be further contacted with an effector molecule under suitable conditions and for sufficient time for conjugation of the effector molecule to the RBC-peptide to form an RBC-peptide-effector molecule conjugate.

This application claims priority from SG10202101003S filed 29 Jan. 2021, the contents and elements of which are herein incorporated by reference for all purposes.

### FIELD OF THE INVENTION

The present invention relates generally to the field of molecular biology. In particular, the present invention relates to methods of modifying cell surface proteins of red blood cells and uses of the same.

### BACKGROUND

Methods for generating surface-modified red blood cells for therapeutic use has been met with many challenges. Most traditional methods, such as chemical functionalization or genetically engineered red blood cells, have to compromise between harsh chemical treatments, that are detrimental to long term survival of red blood cells, and highly expensive genetic engineering and culturing of progenitor cells. Existing enzymatic methods are inefficient without prior genetic engineering. Thus, there is an unmet need to provide efficient methods of generating surface-modified red blood cells for therapeutic use.

The present invention has been devised in light of the above considerations.

### SUMMARY OF THE INVENTION

At its most general, the present disclosure refers to methods of modifying post-enucleated red blood cells, and to modified red blood cells comprising a post-enucleation surface-conjugated effector molecule. In particular, the present disclosure refers to methods of modifying red blood cells that have not been genetically modified.

In one aspect, the present disclosure provides a method comprising:
(a) contacting a Red Blood Cell (RBC) with a peptide or polypeptide in the presence of a ligase, under suitable conditions and for sufficient time to allow ligation of the peptide or polypeptide to the RBC to form an RBC-peptide or RBC-polypeptide conjugate;
wherein the peptide or polypeptide comprises a C-terminal ligase recognition sequence.

The method may further involve a step of washing the RBC-peptide conjugate, such as to remove peptide that is not conjugated to the RBC.

The ligase may be OaAEP1 ligase, such as mutant OaAEP1 ligase, such as OaAEP1-Cys247Ala.

The C-terminal recognition sequence may have a sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

In some cases, the C-terminal recognition sequence does not allow high ligation efficiency by an OaAEP1 ligase. The C-terminal recognition sequence may have the sequence Xaa₁GG, wherein Xaa₁ is any amino acid except G. The C-terminal recognition sequence may have the sequence NG or NCL.

In some aspects, the peptide or polypeptide is an effector molecule. An effector molecule may have a C-terminal recognition sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

Where the RBC is conjugated to an effector molecule, the method may be referred to as a “1-step method”.

In some cases, the peptide is a linker peptide. The linker peptide has a C-terminal ligase recognition sequence. The linker peptide has an N-terminal motif for conjugation to another peptide or polypeptide, such as an effector molecule. The N-terminal motif may be a ligase recognition sequence, a click chemistry functional group, or a biotin moiety.

The ligase recognition sequence may be a recognition sequence for a ligase selected from OaAEP1 ligase, sortase, an asparaginyl peptidase, of butelase 1, or any mutant form or variant thereof, preferably OaAEP-Cys247Ala ligase.

Where the linker peptide has an N-terminal ligase recognition sequence, the sequence may comprise G, GG, GL, GGG, GLG and GGL.

Where the peptide is a linker peptide, ligation of the peptide to the RBC results in the formation of an RBC-linker peptide conjugate.

The RBC-linker peptide conjugate may be contacted with an effector molecule. Such a method may be referred to as a “2-step method”, involving a first step of conjugation to the linker peptide and a second step of conjugation to the effector molecule.

Where the linker peptide has an N-terminal ligase recognition site, the RBC-linker peptide may be contacted with an effector molecule that has a C-terminal ligase recognition sequence. Such contacting may occur in the presence of a ligase, for sufficient time and under suitable conditions to allow ligation of the effector molecule to the linker peptide, thereby forming an RBC-linker-effector molecule conjugate. In such methods, the ligase present during the contacting of the RBC to the linker peptide may be referred to as the first ligase, and the ligase present during the contacting of the RBC-linker conjugate and the effector molecule may be referred to as the second ligase. The first and second ligases may be the same. The first and second ligases may be different. In a particularly preferred method, the first and second ligases are OaAEP1 ligases, preferably OaAEP1-Cys247Ala.

In such cases, the C-terminal ligase recognition sequence of the linker peptide may be referred to as the first C-terminal ligase recognition sequence, and the C-terminal ligase recognition sequence of the effector molecule may be referred to as the second C-terminal ligase recognition sequence. In such methods it is generally advantageous that the first C-terminal ligase recognition sequence and the second C-terminal ligase recognition sequence are not the same. Where the first and second ligases are both the same, it may advantageous that the first C-terminal ligase recognition sequence and the second C-terminal ligase recognition sequence are not the same. In particular, it may be advantageous for the first C-terminal ligase recognition sequence to be less optimised for ligase recognition than the second C-terminal ligase recognition sequence. In other words, the ligation of the RBC to the linker by the first ligase may be less efficient than the ligation of the effector molecule to the RBC-linker peptide. Without wishing to be bound by theory, the variation in ligation efficiency is believed that this reduces self-ligation by the linker. In such methods the first C-terminal ligase recognition sequence has the sequence Xaa₁GG, wherein Xaa₁ is any amino acid except G or has the sequence NG or NCL, and the second C-terminal ligase recognition sequence has a sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

In some cases, the linker peptide comprises a click chemistry functional group at the N-terminus, or otherwise exposed on the linker peptide. The click chemistry functional group may be selected from an azide moiety, a tetrazine moiety, a methyl tetrazine moiety a diarylcytooctyne (DBCO) moiety or a Transcyclooctyne (TCO) moiety.

In some cases, the linker peptide comprises a biotin moiety at the N-terminus, or otherwise exposed on the linker peptide.

Where the linker peptide comprises an N-terminal motif for conjugation to another peptide that is not a ligase recognition motif, such as where the linker peptide comprises a click chemistry functional group of biotin moiety at the N terminus or otherwise exposed on the linker peptide, the C-terminal ligase recognition sequence may be selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL. The choice of N-terminal motif is determined to be complementary to the second C-terminal motif on the effector molecule (GL-).

Where the linker peptide comprises a click chemistry functional group, the RBC-linker conjugate may be contacted with an effector molecule that has a complementary click chemistry functional group, such as a click chemistry functional group at the C-terminal of the effector molecule or otherwise exposed on the effector molecule. The complementary click chemistry functional group may be selected from an azide moiety, a tetrazine moiety, a methyltetrazine moiety a diarylcytooctyne (DBCO) moiety or a Transcyclooctyne (TCO) moiety.

Where the click chemistry functional group of the linker peptide is an azide moiety, the complementary click chemistry functional group of the effector molecule is a DBCO moiety. Where the click chemistry functional group of the linker peptide is a DBCO moiety, the complementary click chemistry functional group of the effector molecule is an azide moiety. Where the click chemistry functional group of the linker peptide is a TCO moiety, the complementary click chemistry functional group of the effector molecule is a tetrazine moiety or a methyltetrazine moiety. Where the click chemistry functional group of the linker peptide is a tetrazine moiety or a methyltetrazine moiety, the complementary click chemistry functional group of the effector molecule is a TCO moiety.

Where the linker peptide comprises a click chemistry functional group, the RBC-linker peptide conjugate is contacted with the effector molecule for sufficient time and under suitable conditions for conjugation of the RBC-linker conjugate to the effector molecule by click chemistry.

Where the linker peptide comprises a biotin moiety, the RBC-linker conjugate is contacted with streptavidin or avidin for sufficient time and under suitable conditions for conjugation of the biotin moiety of the linker conjugate to the streptavidin or avidin, thereby forming an RBC-linker-streptavidin or RBC-linker-avidin conjugate. The RBC-linker-streptavidin or RBC-linker-avidin conjugate may be contacted with a biotinylated effector moiety. A biotinylated effector moiety is an effector moiety that comprises a biotin moiety, such as an effector molecule that has been conjugated to a biotin moiety. The RBC-linker-streptavidin or RBC-linker-avidin conjugate may be contacted with the biotinylated effector moiety for sufficient time and under suitable conditions for conjugation of the biotin moiety of the effector molecule to the streptavidin or avidin moiety of the RBC-linker-streptavidin or RBC-linker-avidin conjugate, thereby forming an RBC-linker-streptavidin-effector molecule conjugate or RBC-linker-avidin-effector molecule conjugate.

As noted above, certain methods disclosed herein are “2-step” methods, involving a first step of conjugation to the linker and a second step of conjugation to the effector molecule.

In one such aspect, the present disclosure provides a method comprising:
(a) contacting an RBC with a linker peptide in the presence of a ligase, under suitable conditions and for sufficient time to allow ligation of the linker peptide to the RBC to form an RBC-linker conjugate;
wherein the linker peptide comprises a first C-terminal ligase recognition sequence and an N-terminal ligase recognition sequence; and
(b) contacting the RBC-linker conjugate from (a) with an effector molecule in the presence of a ligase, under suitable conditions and for sufficient time to allow ligation of the effector molecule to the ligase to form an RBC-linker-effector conjugate; and
wherein the effector molecule comprises a second C-terminal ligase recognition sequence.

The method may involve a step of washing the RBC-linker conjugate. Such wash step is preferably performed before the RBC-linker conjugate is contacted with the effector molecule.

The method may involve a step of washing the RBC-linker-effector molecule conjugate.

In an alternative aspect, the present disclosure provides a method comprising:
(a) contacting an RBC with a linker peptide in the presence of a ligase, under suitable conditions and for sufficient time to allow ligation of the linker peptide to the RBC to form an RBC-linker conjugate; wherein the linker peptide comprises a C-terminal ligase recognition sequence and an N-terminal biotin moiety; and
(b) contacting the RBC-linker conjugate from (a) with streptavidin, wherein the RBC-linker conjugate and the streptavidin are contacted under suitable conditions and for sufficient time to allow conjugation of the streptavidin to bind to the biotin moiety of the linker to form an RBC-linker-streptavidin conjugate; and
(c) contacting the RBC-linker-streptavidin conjugate with a biotinylated effector molecule under suitable conditions and for sufficient time to allow conjugation of the biotinylated effector molecule to the RBC-linker-streptavidin conjugate to form an RBC-linker-streptavidin-effector molecule conjugate.

The method may involve a step of washing the RBC-linker conjugate. Such wash step is preferably performed before the RBC-linker conjugate is contacted with streptavidin.

The method may involve a step of washing the RBC-linker-streptavidin conjugate. Such wash step is preferably performed before the RBC-linker-streptavidin conjugate is contacted with the effector molecule.

The method may involve a step of washing the RBC-linker-streptavidin-effector molecule conjugate.

In another alternative aspect, the present disclosure provides a method comprising:
(a) contacting an RBC with a linker peptide in the presence of a ligase, under suitable conditions and for sufficient time to allow ligation of the linker peptide to the RBC to form an RBC-linker conjugate; wherein the linker peptide comprises a C-terminal ligase recognition sequence and click chemistry functional group; and
(b) contacting the RBC-linker conjugate from (a) with an effector molecule that comprises a complementary click chemistry functional group, wherein the RBC-linker conjugate and the effector molecule are contacted under suitable conditions and for sufficient time to allow conjugation of the effector molecule to the linker by copper-free Click chemistry to form an RBC-linker-effector molecule conjugate.

The method may involve a step of washing the RBC-linker conjugate. Such wash step is preferably performed before the RBC-linker conjugate is contacted with the effector molecule.

The method may involve a step of washing the RBC-linker-effector molecule conjugate.

The click chemistry functional group may be selected from an azide moiety, a tetrazine moiety, a methyl tetrazine moiety a diarylcytooctyne (DBCO) moiety or a Transcyclooctyne (TCO) moiety.

The complementary click chemistry functional group may be selected from an azide moiety, a tetrazine moiety, a methyl tetrazine moiety a diarylcytooctyne (DBCO) moiety or a Transcyclooctyne (TCO) moiety.

Where the click chemistry functional group of the linker peptide is an azide moiety, the complementary click chemistry functional group of the effector molecule is a DBCO moiety. Where the click chemistry functional group of the linker peptide is a DBCO moiety, the complementary click chemistry functional group of the effector molecule is an azide moiety. Where the click chemistry functional group of the linker peptide is a TCO moiety, the complementary click chemistry functional group of the effector molecule is a tetrazine moiety or a methyltetrazine moiety. Where the click chemistry functional group of the linker peptide is a tetrazine moiety or a methyltetrazine moiety, the complementary click chemistry functional group of the effector molecule is a TCO moiety.

Certain methods described herein involve a linker peptide. The linker peptide may comprise a C-terminal ligase recognition sequence for ligation to the Red Blood Cell. The linker peptide may comprise a N-terminal motif for conjugation to another peptide, such as an effector molecule. The N-terminal motif may be a ligase recognition sequence, a click chemistry functional group, or a biotin moiety.

The linker peptide comprises a C-terminal ligase recognition sequence that has a sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL or has the sequence Xaa₁GG, wherein Xaa₁ is any amino acid except G or has the sequence NG or NCL.

The linker peptide has an N-terminal ligase recognition sequence, the sequence may comprise G, GG, GL, GGG, GLG and GGL.

Where the linker peptide has an N-terminal ligase recognition sequence the C-terminal ligase recognition sequence have the sequence Xaa₁GG, wherein Xaa₁ is any amino acid except G or has the sequence NG or NCL.

The linker peptide may have a click chemistry functional group or biotin moiety at the N-terminus, or otherwise exposed on the molecule. The click chemistry functional group may be selected from an azide moiety, a tetrazine moiety, a methyl tetrazine moiety a diarylcytooctyne (DBCO) moiety or a Transcyclooctyne (TCO) moiety.

Where the linker peptide has a click chemistry functional group, or a biotin moiety, the C-terminal ligase recognition sequence may have a sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NC, preferably NGL, NPL or NDL or has the sequence Xaa₁GG, wherein Xaa₁ is any amino acid except G or has the sequence NG or NCL. Preferably, the sequence is selected from NGL, NDL, NPL, NAL, NCL, NEL, NFL, NHL, NKL, NIL, NLL, NQL, NRL, NSL, NTL, NVL, NWL, NYL, NSLD, NSLAN or NG, most preferably NGL, NPL or NDL.

The linker peptide preferably has a linker body sequence between the C-terminal ligase recognition sequence and the N-terminal ligase recognition sequence, click chemistry functional group or biotin moiety. The linker body sequence may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. The linker body sequence may comprise an α-helical peptide sequence. The linker body sequence may comprise repeats of the sequence EAAAK. The linker body sequence may comprise 1-10 repeats of the sequence EAAAK, such as 1, 2, 3, 4, 5, 6, 7, 8, 9 or 10 repeats of the sequence EAAAK, preferably 3 repeats of the sequence EAAAK. The linker body sequence may comprise the sequence EQKLISEEDL.

The linker may comprise or consist of a sequence selected from:

GLGEQKLISEEDLGLPETGG;
DBCO-EAAAKEAAAKEAAAKNGL
(where DBCO refers to diarylcytooctyne);
Azide-GSSGSGGEQKLISEEDLGGSGGSGSGNGL;
GLGEQKLISEEDLGLPETGG;
GGGEQKLISEEDLGLPETGG;
GLGEQKLISEEDLGNGL;
GGGEQKLISEEDLGNGL;
and
GLG(EAAAK)₅LPETGG.

Certain methods disclosed herein involve an effector molecule. The effector molecule may be selected from the group consisting of protein, enzyme, cell-surface marker, monoclonal antibody, single chain antibody, nanobody, therapeutic agent, cytokine, chemokine, antibody fragment and combinations thereof. In some preferred aspects, the effector molecule is a monoclonal antibody, a single chain antibody or a nanobody.

In some aspects, the effector molecule is a cytokine or chemokine. For example, the effector molecule may be IL-8, IL-12, CD137L, IL-15, -IL-7, IL-2, or IL-10. In some aspects, the effector molecule is an immunomodulatory ligand, such as 4-1 BB ligand (4-1BBL).

In some aspects, the effector molecule is an enzyme. For example, the effector molecule may be L-asparaginase, arginine deaminase, uricase or other enzyme known to be useful in an enzyme replacement therapy.

In some aspects, the effector molecule is an antibody, such as a single chain antibody, nanobody, monoclonal antibody or antigen binding fragment. For example, the effector may be raised against a target of interest such as a cancer cell marker such as a leukemic cell marker. Markers include CXCR4/CD33, EGFR, HER2 or another cancer cell surface protein. The effector may be raised against a toxin such as botulinum toxin, or against a pathogen such as a bacteria or virus.

The effector molecule may comprise a C-terminal ligase recognition molecule. The C-terminal ligase recognition molecule is preferably a sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

The effector molecule may be a biotinylated effector moiety. A biotinylated effector moiety is an effector moiety that comprises a biotin moiety, such as an effector molecule that has been conjugated to a biotin moiety. The biotin moiety is preferably exposed on the effector moiety, such that it is available for conjugation to avidin or streptavidin.

The effector molecule may comprise a complementary click chemistry functional group. The complementary click chemistry functional group may be selected from an azide moiety, a tetrazine moiety, a methyl tetrazine moiety a diarylcytooctyne (DBCO) moiety or a Transcyclooctyne (TCO) moiety. The complementary click chemistry functional group is complementary to the click chemistry functional group of the linker peptide. Where the click chemistry functional group of the linker peptide is an azide moiety, the complementary click chemistry functional group is a DBCO moiety. Where the click chemistry functional group of the linker peptide is a DBCO moiety, the complementary click chemistry functional group is an azide moiety. Where the click chemistry functional group of the linker peptide is a TCO moiety, the complementary click chemistry functional group is a tetrazine moiety or a methyltetrazine moiety. Where the click chemistry functional group of the linker peptide is a tetrazine moiety or a methyltetrazine moiety, the complementary click chemistry functional group is a TCO moiety.

The effector molecule may have a size of at least 10 kDa. For example, the effector molecule may be an antibody or antigen binding fragment that has a size of at least 10 kDa. The effector molecule may have a size of at least 10 kDa, at least 10.5 kDa, at least 11 kDa, at least 11.5 kDa, at least 12 kDa, at least 12.5 kDa, at least 13 kDa, at least 13.5 kDa, at least 14 kDa, at least 14.5 kDa, at least 15 kDa, at least 16 kDa, at least 17 kDa, at least 18 kDa, at least 19 kDa, at least 20 kDa, at least 21 kDa, at least 22 kDa, at least 23 kDa, at least 24 kDa or at least 25 kDa.

The effector molecule may have a size of at least 7 kDa. For example the effector molecule may be a small protein or polypeptide with a size of at least 7 kDa. The effector molecule may have a size of at least 7 kDa, at least 7.5 kDa, at least 8 kDa, at least 8.5 kDa, at least 9 kDa, at least 9.5 kDa, at least 10 kDa, at least 10.5 kDa, at least 11 kDa, at least 11.5 kDa or at least 12 kDa.

Methods disclosed herein may involve a ligase selected from the group consisting OaAEP1 ligase, Sortase A, an asparaginyl peptidase, of butelase 1, or any mutant form or variant thereof, preferably OaAEP-Cys247Ala ligase. Where the method is a two-step method, involving conjugation of a linker peptide to an RBC and conjugation of an effector molecule to an RBC-linker conjugate, the ligase for conjugation of the linker peptide to the RBC (the first ligase) and the ligase for conjugation of the effector molecule to the RBC-linker conjugate (the second ligase) may be the same or may be different. In preferred aspects, the first and second ligases are the same. In particularly preferred aspects, the first and second ligases are OaAEP1 ligases, preferably OaAEP1-Cys247Ala ligases.

In some methods described herein, the RBC is a deglycosylated RBC. In other words, the RBC has been previously treated to remove carbohydrate from glycoproteins in the RBC membrane. The RBC may have been enzymatically deglycosylated with a glycosidase selected from the group consisting PNGaseF, EndoH, O-glycosidase and exoglycosidases (Mannosidase, neuraminidase and β-N-Acetylhexosaminidase). For example, a step of contacting the red blood cell with PNGaseF, EndoH, O-glycosidase or an exoglycosidase (Mannosidase, neuraminidase and/or β-N-Acetylhexosaminidase). In some methods provided herein, a step of deglycosylating the red blood cell occurs prior to any step of contacting the red blood cell with an effector molecule or linker peptide. Methods disclosed herein may involve contacting a degylcosylated red blood cell with a peptide, such as contacting a deglycosylated red blood cell with an effector molecule or a linker peptide.

The present disclosure also describes modified red blood cells produced by the methods disclosed herein, and uses therefore.

The modified red blood cell may comprise, on its exterior surface, a peptide, wherein the peptide is conjugated to a native red blood cell membrane protein. The peptide may be an effector molecule. In such cases, the effector molecule may be directly conjugated to the membrane protein. The peptide may be a linker peptide. In such cases, an effector molecule may be conjugated to the linker peptide. Thus, in some aspects, the disclosure provides a modified red blood cell comprising, on its exterior surface, an effector molecule, wherein the effector molecule is conjugated to a native red blood cell membrane protein via a linker peptide. The linker peptide may be any suitable linker peptide, such as the linker peptides described above. The effector molecule may be any suitable effector molecule, such as the effector molecules described above. The modified red blood cell may be a deglycosylated red blood cell.

In an aspect, the present disclosure provides a modified red blood cell for use in medicine, the use of a modified red blood cell in the manufacture of a medicament for treating a disease or disorder, or a method of treatment comprising administering a modified red blood cell to an individual or patient in need of treatment. The treatment may be a treatment for an enzyme deficiency, metabolic disease, immune-related disorder, blood disorder, or cancer.

In another aspect, the present disclosure refers to a method of surface-modifying a native red blood cell post-enucleation comprising, exposing the native red blood cell obtained from a subject to an effector molecule, conjugating the effector molecule to the red blood cell, thereby modifying the red blood cell and a modified red blood cell obtained by the method disclosed herein, a red blood cell as disclosed herein for use in therapy, and the use of the modified red blood cell as disclosed herein in the manufacture of a medicament treating a disease or disorder.

The invention includes the combination of the aspects and preferred features described except where such a combination is clearly impermissible or expressly avoided.

### SUMMARY OF THE FIGURES

The invention will be better understood with reference to the detailed description when considered in conjunction with the non-limiting examples and the accompanying drawings, in which:

FIG. 1 OaAEP1 Cys247Ala can be used to covalently ligate peptides on the human red blood cell surface. (A) Western Blot analysis of human RBCs (hRBCs) ligated with a biotinylated peptide (B-peptide/B-TL5), detected using Streptavidin-HRP. (B) A comparison of B-peptide (monobiotinylated) on human red blood cell (hRBC) ligation products with a serial dilution of dibiotinylated HRP standard, indicating a quantification of ligated peptide per human red blood cell (hRBC). In A and B, molecular weights (kDa) of protein markers are shown on the left. (C) Flow cytometry analysis of B-peptide ligation to hRBCs detected using streptavidin-AF647 (mean±SEM, n=3 red blood cells from 3 donors). (D) Immunofluorescence images of hRBCs incubated or ligated with the peptide (stained green using PE-anti-biotin antibody), representing colocalization of the peptide on the cell membrane (stained red using CellMask™ deep red). (E) Mean fluorescence of PE-anti-biotin per unit cellular area in ligated and control hRBCs. Student's t-test ***P<0.001.

FIG. 2 Conjugation of nanobodies to human red blood cells using a two-step ligation method. (A) Outline of the conjugation approach, where a linker peptide is used to facilitate the ligation of a protein. (B) Flow cytometry analysis of FLAG tag on EGFRVHH that was ligated to human red blood cells (mean±SEM, n=3 independent replicates). (C) Immunofluorescence images of human red blood cells (hRBCs) incubated or ligated with EGFR VHH (stained green using AF488-FLAG tag antibody), showing the extent of colocalization with the cell membrane (stained red using CellMask™ deep red). (D) Mean AF488 fluorescence per unit hRBC area. Student's t-test ***P<0.001.

FIG. 3 Conjugation of large proteins to human red blood cells (hRBCs) via a streptavidin linker. (A) Outline of the streptavidin-mediated conjugation approach used to conjugate large proteins on the human red blood cells (hRBC) surface. (B) Flow cytometry analysis of a biotinylated anti-his-tag monoclonal antibody (B-His-mAb) that was conjugated to hRBCs, presented as mean±SEM (n=3). (C) Immunofluorescence images of human red blood cells incubated or conjugated with B-His-mAb (stained green using a secondary donkey-anti-rabbit AF488 antibody), displaying the extent of colocalization with the cell membrane (stained red using CellMask™ deep red). (D) Mean intensity of AF488 signal per unit cellular area derived from the cell mask signal of the human red blood cells shown in (C). (E) Mean fluorescent intensity of FLAG-tag staining of B-His-mAb conjugated red blood cells that were used to pulldown proteins bearing both FLAG and his tags. (F) Images of biotinylated-HRP (B-HRP) conjugated red blood cells and unmodified red blood cells after an incubation with 3,3′-Diaminobenzidine (DAB) chromogen followed by H&E staining. Horseradish peroxidase activity was visualized by the formation of the characteristic brown precipitate. (G-H) Flow cytometric analysis of biotinylated human interleukin 8 (hIL-8) conjugation on hRBCs. hIL-8 was detected on the surface of hRBCs using a primary mouse-anti-hIL8 antibody which was subsequently detected using a donkey anti-mouse AF647 antibody. (I) Effect of L-Asparaginase conjugated hRBCs on Sup-B15 leukemic cell viability assessed using CCK8 assay following 4 days of co-culture at a ratio of 1 RBC: 5 leukemia cells (corresponding to 200,000 RBCs/mL). Purified biotinylated L-Asparaginase at a concentration of 5 IU/mL is included as a positive control. G and H were conducted on hRBCs from separate donors (presented as mean±SEM, n=3). Student's t-test ***P<0.001.

FIG. 4 Bio-orthogonal, covalent conjugation of large molecules to human red blood cells via enzymatic ligation and click chemistry. (A) Outline for the conjugation of molecules on the red blood cell surface using copper-free click chemistry. (B) Flow cytometry analysis of CalFluor-647, an azido-molecule that fluoresces only upon clicking with a DBCO peptide that was ligated to human red blood cells (hRBCs). (C) Flow cytometry analysis of a biotinylated azido-peptide (B-TK3-N3) or an azido antibody (CMTM6-mAb-N3) that were clicked on DBCO-peptide ligated human red blood cells, detected using streptavidin-AF647 and donkey-anti-mouse-AF647 antibody. (D) Immunofluorescent images of hRBCs that were conjugated with azido-monoclonal antibodies using click chemistry. The human red blood cell membrane is shown in green (CellMask Green) while the azido-antibody is detected using a donkey-anti-mouse AF647 antibody (red). (E) Efficiency of conjugation of monoclonal antibodies on human red blood cells using copper-free click chemistry represented as the extent of colocalization of the AF647 signal with CellMask. (F) GFP conjugation on hRBCs using the combinatorial enzymatic/click chemistry approach, assessed using flow cytometric analysis of conjugated and control hRBCs. (G) Representative flow cytometry histograms for the data in F. B, E and F were conducted on hRBCs from separate donors (presented as mean±SEM, n=3). Student's t-test ***P<0.001.

FIG. 5 Surface modification of mouse red blood cells (mRBCs) using OaAEP1 ligase. (A) Flow cytometry analysis of a biotinylated peptide (B-Peptide/B-TL5) that was conjugated to mRBCs using OaAEP1 ligase, detected using streptavidin AF647. (B) Immunofluorescent images of mRBCs incubated or ligated with a biotinylated peptide. The ligated peptide is stained green (PE-anti-biotin antibody), and the images display the degree of colocalization with the cell membrane (stained red using CellMask™ deep red). (C) Mean PE fluorescence per unit cellular area of biotinylated peptide ligated on the mRBC membrane as shown in (B). (D) Comparison of the effect of peptide ligation motif on ligation yield assessed using flow cytometry. mRBCs were ligated with Biotin-(EAAAK)₃—X peptides where X represents the indicated C-terminal recognition motif. Data is shown from 3 biological replicates using blood obtained from three separate donors. (E) Effect of peptide length on ligation yield assessed using flow cytometric analysis of Biotin-(EAAAK)ₙ-NGL, where n represents the number of EAAK repeats. The graph represents data from 3 biological repeats. Student's t-test ***P<0.001.

FIG. 6 Conjugation of red blood cells (RBCs) is efficient and versatile. (A) Flow cytometry analysis of RBC-B-peptide ligation (human and mouse) at different time points. The reaction was quenched at each time point and stained with streptavidin-Alexa Fluor 647 to detect intensity of biotinylated peptides ligated on the red blood cell surface. (B) Flow cytometry analysis of click chemistry-mediated conjugation of a biotinylated azido-peptide (B-TK3-N3), assessing the effect of azido-peptide concentration and reaction time on yield of conjugated peptide (detected using streptavidin-Alexa Fluor 647). (C) Flow cytometry to verify the ability to perform click chemistry with reversed orientations of the functional groups. Human red blood cells (hRBCs) were ligated with an azido-peptide (GL29) and clicked with a DBCO-labelled FLAG-tag-containing peptide (GK25) (Student's t-test ***P<0.001). Human red blood cells (hRBCs) were subsequently stained with an anti-FLAG tag antibody prior to analysis.

FIG. 7 Conjugated red blood cells are biocompatible and stable in vivo. (A) Flow cytometry analysis of Annexin V staining of unmodified or biotinylated peptide (B-TL5) ligated mouse and human RBCs. (B) Percentage of CFSE-stained mouse red blood cells (mRBCs) that were unmodified or ligated with B-peptide (B-TL5) remaining in the circulation of NSG-S mice over a period of 24 hours, determined based on a flow cytometry analysis of CFSE. (C) Stability of ligated biotinylated peptides on the mRBC surface in the circulation of NSG-S mice over a period of 24 hours represented as the mean fluorescent intensity of Streptavidin-AF647 staining on engineered red blood cells. (D) Representative images of blood smears taken from mice injected with PBS, unmodified or B-peptide ligated CFSE-labelled mouse red blood cells (mRBCs), 24 hours post administration. (E) Mean streptavidin AF647 fluorescence per unit cellular area of externally administered mouse red blood cells (mRBCs) for biotinylated peptide ligated and unmodified mouse red blood cells (mRBCs) for blood smears taken from mice 24 hours post administration.

FIG. 8 (A) Outline of the experiment performed to determine in vivo stability and half-life of engineered mRBCs. (B) Percentage of CFSE-stained mRBCs that were unmodified or ligated with B-peptide (B-TL5) remaining in the circulation of NSG-SGM3 or C57BL/6 mice over a period of 24 hours, determined based on a flow cytometry analysis of CFSE. (C) Stability of ligated biotinylated peptides on the mRBC surface in the circulation of NSG-SGM3 or C57BL/6 mice over a period of 24 hours represented as the mean fluorescent intensity of Streptavidin-AF647 staining on engineered mRBCs.

FIG. 9 (A) Comparison of the effect of peptide ligation motif on ligation yield assessed using flow cytometry. hRBCs were ligated with Biotin-(EAAAK)ₛ-X peptides where X represents the indicated C-terminal recognition motif. Data is shown from 3 biological replicates using blood obtained from three separate donors. (B) Effect of peptide length on ligation yield assessed using flow cytometric analysis of Biotin-(EAAAK)ₙ-NGL, where n represents the number of EAAK repeats. The graph represents data from 3 biological repeats. Student's t-test ***P<0.001.

FIG. 10 (A) Representative flow cytometry histograms illustrating the relative efficiency of EGFR VHH ligation on hRBCs with or without the GN20 peptide, B-GN20 (a biotinylated form of GN20 which prevents ligation at the N-terminal), and TL20 (a scrambled peptide of similar length). Sequences for each linker peptide are indicated on the right. (B) Percentage of VHH-ligated hRBCs and mean fluorescent intensity of the FLAG-tag signal of hRBCs for each of the conditions from A (mean±SEM. n=3 independent replicates). (C) Effect of different linker peptides with varying combinations of N- and C-terminal motifs assessed for their ability to facilitate 2-step ligation. Data is represented as the mean fluorescent intensity of the FLAG tag signal in the hRBC population following flow cytometric analysis. Peptide sequences for each peptide are indicated on the left of the graph. The presence of FLAG-tagged VHH was detected using an anti-FLAG tagged monoclonal antibody conjugated with APC. Student's t-test ***P<0.001.

FIG. 11 (A) Outline of the conjugation approaches utilized for efficient conjugation of proteins on hRBCs, where either a linker peptide is used to facilitate the ligation of a protein or deglycosylation is carried out prior to direct ligation of the protein. (B) Flow cytometric analysis of hRBCs for FLAG-tag signals following direct ligation with EGFR VHH on unmodified hRBCs, or following the treatment with a cocktail of glycosidases (deglycosylated). Specific glycosidases used are indicated for each condition. PNGase F and Endo H cleave N-glycans, 0-glycosidase removes O-glycans while exoglycosidases remove individual monosaccharides (Mannosidase, neuraminidase and β-N-Acetylhexosaminidaset. (C) Flow cytometric analysis of hRBCs of EGFR VHH ligation on hRBCs following selected sequential ligation/deglycosylation steps in different orders. (D) Pulldown of EGFR-positive 4T1-tdTomato cells using hRBCs conjugated with VHH EGFR using either direct ligation or linker peptides. Pulldown efficiency of 4T1-tdTomato-hEGFR cells was detected using western blot, probing for EGFR, while HBA was used as an internal control for RBCs. (E) Relative pulldown efficiency of 4T1-tdTomato cells via VHH EGFR ligated or control RBCs measured by quantifying the tdTomato fluorescence in pulldown lysates. For G and H, the presence of FLAG-tagged VHH was detected using an anti-FLAG tagged monoclonal antibody conjugated with APC. Student's t-test ***P<0.001.

FIG. 12 Biotin pulldown experiment coupled with LFQ-Mass Spectrometry to identify candidate proteins on RBCEVs ligaed with OaAEP1 ligase performed on biotinylated peptide-ligated RBCEVs derived from human RBCs. Circles 5, 7, 8, 11, 13 and 27 are proteins of 25-50 kDa in RBCEVs associated with the biotinylated peptide.

FIG. 13 Cross-comparison of this candidate proteins from RBCEVs with existing RBC membrane proteome studies assessing their relative abundance and estimated copy number.

FIG. 14 Comparison of ligation efficiency using different enzymes (OaAEP1 Cys247Ala or Sortase A heptamutant). A range of biotinylated peptides with identical EK15 sequences (EAAAKEAAAKEAAAK) and different only in their C-terminal motifs (either optimized in this study (-NPL for OaAEP1)) or previously reported to be compatible with each enzyme (LPETGGG for Sortase A, -NGL for OaAEP1) were ligated on hRBCs and the ligation efficiency assessed using flow cytometry. Short peptides of different sequence and length are included as positive controls. Ctrl hRBCs denotes hRBCs simply incubated with the enzymes in the absence of any peptide.

### DETAILED DESCRIPTION OF THE INVENTION

Allogeneic cell therapies, such as engineered red blood cells (RBCs) and extracellular vesicles (EVs), have recently come to light as potential drug carriers, after years of being ignored for more artificial and amenable drug delivery systems such as liposomes and DNA polyplexes (Chatin et al., 2015; Durymanov & Reineke, 2018). While therapeutics encapsulated in red blood cells have seen rapid development in clinical trials, their application is limited by the selectively permeable nature of the red blood cell plasma membrane.

The solution to this is the use of surface functionalized red blood cells, where therapeutic molecules are immobilized on the surface of red blood cells. However, current methods of generating surface engineered red blood cells have been met with difficulty in establishing a balance between biocompatibility, efficiency and scalability. Most surface modification methods currently in use involve either harsh chemical treatments or genetic modifications to hematopoietic progenitor cells which are then differentiated in vitro, which is a very costly and time-consuming process. In the first instance, even the more biocompatible chemical modification methods have been shown to decrease in vivo half-life, due to red blood cells surface proteins being compromised due to, for example, decay accelerating factor, leading to eventual lysis by complement. An enzymatic approach was also presented by Shi et al. (Shi et al, 2014), where Sortase A was used to ligate peptides onto the surface of genetically engineered red blood cells expressing recognition motifs for Sortase A. This allowed the biocompatible ligation of peptides onto the surface of the genetically engineered red blood cells, leading to the development of a red blood cell engineering platform. However, the genetic engineering method requires a tedious and expensive procedure of stem cell culture and differentiation. More recently, it was found that mature red blood cells can be conjugated directly with peptides using Sortase A (Pishesha et al, 2017). However, covalent conjugation of mature red blood cells with proteins is not yet demonstrated. In addition, there have been contributions to the field in the form of affinity-based red blood cell-binding molecules and lipid insertion. However, these all suffer from the transient nature that is characteristic of these methods (Villa et al, 2016; Villa et al, 2017).

The present disclosure describes a biocompatible enzymatic method, site-specific surface functionalised red blood cells that maintain high copy numbers of stably conjugated peptides/proteins (for example, but not limited to, monoclonal antibodies, single domain antibodies, enzymes, functional proteins and the like) per cell, bypassing any prior chemical or genetic modifications. The resulting engineered red blood cells maintain the functionality of conjugated proteins and show no sign of toxicity post-conjugation. This enzymatic approach has further been extended by combining it with other methods, such as, but not limited to, bioorthogonal click chemistry and Streptavidin-mediated conjugation, to further increase the versatility and functionality of the engineered red blood cells. These engineered red blood cells provide a more scale-able method to genetically engineered red blood cells and chemically modified red blood cells. These modified red blood cells are then applied in pre-clinical development for a range of diseases including, but not limited to, enzyme replacement therapies, cell-based immunotherapies and other treatments, prophylactic and curative.

In one aspect, there is disclosed a method of surface-modifying a red blood cell post-enucleation comprising, exposing a native red blood cell, conjugating the effector molecule to the red blood cell, thereby modifying the red blood cell. In one example, the red blood cell is a native red blood cell. In another example, the (native) red blood cells are obtained from humans. In yet another example, the red blood cell has not been genetically engineered or modified. It is understood that genetically modified red blood cells are different from native red blood cells, and that a person skilled in the art would be able to differentiate between the two.

In one aspect, the methods disclosed herein are used to generate/modify native red blood cells with proteins, which contain at least 100 amino acid, including, but not limited to, single domain antibodies (sdAB). In another example, the native red blood cells, as described herein, are enzymatically modified on the cell surface. In another example, the effector molecule is a peptide comprising 20 or more amino acids. In one example, the linker is a peptide. In another example, the linker described herein has one end suitable for a sortase reaction and the other end suitable for a protein ligase reaction

Presented herewith are surface-modified red blood cells generated using an approach which provides a balance between biocompatibility, efficiency, stability, speed and scalability. These surface engineered red blood cells are modified primarily using, but not limited to, protein ligases (for example, but not limited to, butelase 1, OaAEP1, variants and mutants of the same), and as such suffer no adverse effects from the process. The ligation allows the stable incorporation of proteins, peptides, monoclonal antibodies or other functional groups onto the RBC membrane at high copy numbers. Also disclosed herein is a two-step ligation, biorthogonal copper-free click chemistry and streptavidin mediated conjugation of biotinylated molecules to extend the versatility of this approach and conjugate a wide range of therapeutic proteins onto the red blood cell surface, while maintaining the biocompatible profile established throughout this workflow. In particular, the combinatorial approach utilizing enzymatic ligation in conjunction with copper-free click chemistry results in wholly biocompatible, covalent, site-specific conjugation of any azide-tagged molecule of interest onto the red blood cells surface at high copy number. In one example, the red blood cell as disclosed herein is attached to an effector molecule by way of a linker. In another example, the red blood cell is attached to a linker, which in turn is attached to an effector molecule.

The procedures used herein to generate these surface functionalised red blood cells are quick (can be completed within 3 to 5 hours, depending on the molecule of interest), easily scalable (blood can be obtained readily from blood banks/patients, and the other reagents can be easily produced in-house), and consistently yield high copy numbers of the molecules of interest. Conjugated molecules are stable, and remain intact and functional on the red blood cell surface, both in vitro and in vivo. As such, this platform provides an alternative for the treatment of a range of diseases such as, but not limited to, enzyme deficiencies (for example, via the immobilization of enzymes on the red blood cell surface, thereby increasing half-life and decreasing the need for frequent dosing), immune related disorders (for example, antigen presentation via artificial red blood cell-based APCs), metabolic diseases (such as familial hypercholesterolemia, Gaucher disease, Hunter syndrome, Krabbe disease, Maple syrup urine disease, Metachromatic leukodystrophy, Mitochondrial encephalopathy, lactic acidosis, stroke-like episodes (MELAS), Niemann-Pick, Phenylketonuria (PKU), Porphyria, Tay-Sachs disease, and Wilson's disease), blood disorders (for example, anaemia of chronic disease, aplastic anaemia, erythrocytosis, hemochromatosism, hypercoagulable disorder, immune thrombocytopenic purpura, iron deficiency anaemia, leucocytosis, leucopenia, polycythemia vera, sickle cell anaemia, and thrombotic thrombocytopenic purpura) and cancer (for example, tumour starvation via enzymes such as asparaginase on the surface of red blood cells).

In one aspect, a native red blood cell is conjugated to a biotinylated peptide. In another example, the red blood cell is conjugated to B-TL5. In another example, the red blood cell is conjugated to a linker, which is conjugated to camelid-derived single domain antibodies (about 15 to 30 kDa). In another example, the red blood cell is conjugated to a linker, which is conjugated to an anti-EGFR single domain antibody. In yet another example, the red blood cell is a biotinylated anti-his tag monoclonal antibody-conjugated human red blood cell. In another example, the red blood cell is conjugated with a DBCO-tagged peptide (EK18).

Red blood cells can be stably conjugated with a range of antibodies and/or proteins at high copy number and maintain functionality after conjugation.

Existing methods of stable surface modification that result in high copy number of large protein molecules employ either chemical or genetic manipulation of red blood cells/progenitor cells, that is both detrimental to red blood cell half-life and function or involves the costly and time-consuming manipulation of progenitor cells. This is the first instance where an enzymatic method is able to meet the versatility and high copy number that was previously only available following extensive genetic/chemical manipulation. The extension of this approach using click chemistry and/or streptavidin further serves to increase functionality of surface functionalized red blood cells. In particular, copper-free click chemistry in combination with enzymatic ligation allows site-specific, bio-orthogonal, covalent conjugation of proteins at high copy number on the red blood cell surface with no detectable adverse or immunogenic effects or decrease to in vivo half-life.

Surface engineered red blood cells are not compromised in their functionality and the process of generating engineered red blood cells uses biocompatible methods such as enzymatic protein ligation, biorthogonal click chemistry and strong affinity interactions.

This method utilizes highly biocompatible methods for surface functionalization, avoiding any risk of damage or detrimental treatment to the red blood cells during the processing stages. This provides an advantage over other methods that need to utilize chemical modification, be it to introduce functional groups onto the red blood cell membrane for further processing or entire proteins. Previous data has displayed that chemical modification methods such as direct chemical biotinylation of red blood cells can compromise Decay accelerating Factor (DAF) on red blood cells, leading to lysis by complement.

Conjugated moieties are stable on the surface of red blood cells in vivo for prolonged periods of time, similar to that of unmodified red blood cells.

Not only do these engineered red blood cells display an in vivo half-life similar to unmodified red blood cells, these modified red blood cells also maintain the conjugated moieties in vivo, to a certain extent protecting them from degradation. Moreover, the covalent and stable nature of the conjugation means that the conjugated moieties don't shed off the red blood cell surface overtime. This is an advantage over peptide/antibody mediated affinity-based conjugation methods, where the conjugated molecule tends to detach from the RBCs over time.

This method is cheaper, faster and simpler than any existing method for red blood cells surface modification that maintains the efficiency and biocompatibility. Moreover, it is easily scaled up. Blood can be obtained from the donor a few hours prior to treatment, washed and fully surface functionalised red blood cells obtained within 3 to 5 hours. These red blood cells can be safely administered back to the patient, in a method similar to a blood transfusion. The lack of potentially toxic chemicals and/or genetic modification makes this method much more readily translatable to the clinic.

Red blood cells need to be obtained from humans. This may be from the intended recipient or from any other individual who is a compatible blood donor (Group O− blood donors preferred)

The methods disclosed in the prior art require the use of genetically engineered red blood cells for efficient sortagging (meaning, sortase-mediated surface modification), especially of larger protein molecules such as nanobodies. The method disclosed in this application does not. Methods disclosed in the art also include only the enzymatic conjugation of peptides to unmodified cells, a method that had been demonstrated before with sortase. Thus, in one example, the method disclosed herein generates surface modified red blood cells without the use of sortase. In another example, sortase is not used for conjugation purposes.

However, the prior art has not employed enzymatic methods for the conjugation of larger functional molecules, such as for example, enzymes. Some methods disclosed in the prior art disclose the direct biotinylation of red blood cells using chemical methods, which have been shown to have adverse effects on red blood cells, especially for long term in vivo studies.

Current enzyme replacement therapies rely on frequent administration of purified enzymes to replace missing enzymes. These enzymes have a very short half-life and are quickly lost from circulation. However, the present conjugation method provide a half-life comparable to that of mature red blood cells (between 30 to 90 days), which is significantly longer than existing methods.

Prophylactic/neutralizing therapies using antibody/decoy receptor-coated red blood cells are capable of binding and neutralizing antigens/viruses/toxins. However such therapies have never reached the clinical trial stage due to low efficacy and high costs. The present method overcomes said limitations.

Also contemplated in the scope of the present application is the use of cell-based therapies to activate the immune system. One such strategy involves using red blood cells as antigen-presenting cells (APCs). This platform can be used to present antigenic molecules in conjunction with co-stimulatory molecules to activate specific arms of the immune system.

Based on the method disclosed herein, red blood cells can be stably conjugated, for example, with a range of antibodies and/or proteins at high copy number and maintain functionality after conjugation. The extension of this approach using click chemistry and/or streptavidin further serves to increase functionality of surface functionalized red blood cells. Copper-free click chemistry combined with enzymatic ligation allows site-specific, bioorthogonal, covalent conjugation of proteins at high copy number on the red blood cells surface, with little to no detectable adverse or immunogenic effects or decrease to in vivo half-life.

Surface-engineered red blood cells utilize biocompatible methods such as enzymatic protein ligation, bioorthogonal click chemistry and strong affinity interactions for surface functionalization, avoiding any risk of damage or detrimental treatment to the red blood cells during the processing stages.

Enzyme replacement therapy: Current enzyme replacement therapies rely on frequent administration of purified enzymes to replace missing enzymes. These have a very short half-life and are quickly lost from circulation. They are also frequently conjugated with molecules that increase half-life such as PEG. However, they only provide a small extension to the in vivo half-life of the drugs. Conjugation to red blood cells instead would provide a half-life comparable to that of mature red blood cells (between 30 to 90 days) which is significantly longer than existing methods. While there are many red blood cell-based therapies in clinical trials for enzyme replacement therapy, all of them involve encapsulating the enzyme inside the red blood cell. This limits the application to enzymes whose substrate can enter red blood cells naturally (via existing endogenous transporters on the red blood cell membrane). The engineered red blood cells described in this disclosure have no such limitations, broadening the scope of red blood cell-based enzyme replacement therapies. Enzymes known to be useful in enzyme replacement therapies include asparaginase, arginine deaminase and uricase.

Prophylactic/neutralizing therapies: Antibody/decoy receptor coated red blood cells capable of binding and neutralizing antigens/viruses/toxins. While certain groups have presented proof of concept of this application, none have reached clinical trials, mostly limited by low efficacy and high costs. The simple and efficient approach presented in this disclosure could overcome some of these limitations.

Immune regulation: Many groups are investigating the use of cell-based therapies to activate the immune system. One such strategy involves using red blood cells as antigen-presenting cells (APCs). This platform can be used to present antigenic molecules in conjunction with costimulatory molecules to activate specific arms of the immune system. Methods disclosed herein involve red blood cells. The methods may involve a step of providing a red blood cell or red blood cells. The methods may involve providing a sample of whole blood and preparing a sample of red blood cells from the whole blood.

Preferably, the RBCs are derived from a human or animal blood sample or red blood cells derived from primary cells or immortalized red blood cell lines. The blood cells may be type matched to the patient to be treated, and thus the blood cells may be Group A, Group B, Group AB, Group O or Blood Group Oh. Preferably the blood is Group O. The blood may be rhesus positive or rhesus negative. In some cases, the blood is Group O and/or rhesus negative, such as Type O−. The blood may have been determined to be free from disease or disorder, such as free from HIV, sickle cell anaemia, malaria. However, any blood type may be used. In some cases, the RBCs are autologous and derived from a blood sample obtained from the patient to be treated. In some cases, the RBCs are allogeneic and not derived from a blood sample obtained from the patient to be treated.

The sample may be a whole blood sample. Preferably, cells other than red blood cells have been removed from the sample, such that the cellular component of the sample is red blood cells.

The red blood cells in the sample may be concentrated, or partitioned from other components of a whole blood sample, such as white blood cells. Red blood cells may be concentrated by centrifugation. The sample may be subjected to leukocyte reduction, such as by leukoreduction filter. The sample may be treated to remove plasma and platelets, such as by washing such as PBS washing.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells.

The obtained RBCs may be subject to further processing, such as washing, tagging, and optionally loading.

In particular, the RBCs may be deglycosylated.

The methods disclosed herein result in the production of modified red blood cells. Such red blood cells are modified on their exterior surface. These may be referred to as surface modified red blood cells, surface functionalized red blood cells or modified red blood cells. The terms are used interchangeably herein.

Methods disclosed herein relate to the conjugation of effector molecules to RBCs. The term “conjugation” refers to the joining of the effector molecule to the RBC. The conjugation may be direct (i.e. the effector molecule is connected to the RBC without any linker) or indirect (i.e. the effector molecule is connected to the RBC by a linker). The conjugation may result in covalent bond formation. The conjugation may be a ligation, such an enzymatic ligation catalysed by a ligase. The conjugation may be a biotin-streptavidin interaction. The conjugation may result from click chemistry.

As used herein, the term “click chemistry” refers to a concept in chemical synthesis, wherein “click” chemistry refers to a class of biocompatible small molecule reactions commonly used in bio-conjugation, allowing the joining of substrates of choice with specific biomolecules. It is of note that click chemistry is not understood to be a single specific reaction, but instead describes a way of generating products that follow examples in nature, which also generates substances by joining small modular units. Examples of uses of click reactions include, but are not limited to, joining a biomolecule and a reporter molecule. Click chemistry is not limited to biological conditions: the concept of a “click” reaction has been used in pharmacological and various biomimetic applications. However, click chemistry has been made notably useful in the detection, localization and quantification of biomolecules. Methods disclosed herein use biocompatible forms of click chemistry that are generally referred to as Copper-free click chemistry.

Click reactions typically occur in one pot, are not disturbed by water, generate minimal and inoffensive by-products, and are characterised by a high thermodynamic driving force that drives it quickly and irreversibly to high yield of a single reaction product, with high reaction specificity (in some cases, with both regio- and stereo-specificity). These qualities make click reactions particularly suitable to the problem of isolating and targeting molecules in complex biological environments. In such environments, products accordingly need to be physiologically stable and any by-products need to be non-toxic (for example, for use in in vivo systems).

Methods of Click chemistry useful in the methods disclosed herein are strain-promoted alkyne-azide cycloaddition (SPAAC) and Inverse electron demand Diels-Alder (IEDDA). SPACC may involve complementary click chemistry functional groups diarylcyclooctyne (DBCO) and azide. IEDDA may involve complementary click chemistry functional groups Transcyclooctyne (TCO) and tetrazine or methyltetrazine.

By developing specific and controllable bioorthogonal reactions (that is, any chemical reaction that can occur inside of living systems without interfering with native biochemical processes), click chemistry has been adapted for use in live cells, for example, using small molecule probes that find and attach to their targets by click reactions. In short, click chemistry describes reactions that are high yielding, wide in scope, create only by-products that can be removed without chromatography, are stereospecific, simple to perform, and can be conducted in easily removable or benign solvents.

Methods described herein involve contacting red blood cells with a linker or effector molecule in the presence of a ligase. By “contacting” we mean bringing each component into proximity close enough to allow conjugation of the components. The terms “contacting” and “incubating” are used interchangeably. The contacting may be performed at room temperature, such as around 20° C. The contacting is preferably performed in the presence of a ligase. The temperature under which the contacting is performed may be optimized according to the temperature for optional functionality of the ligase. Preferably the ligase is an OaAEP1 ligase, and the temperature is about 25° C. The contacting may be performed for a period of time suitable for the conjugation to occur, such as for the ligase to catalyse the conjugation. The suitable time period will be readily appreciable to the skilled person, and may involve contacting for 1 day, less than 1 day, 12 hours, or less than 12 hours, such as about 12 hours, about 11 hours, about 10 hours, about 9 hours, about 8 hours, about 7 hours, about 6 hours, about 5 hours, about 4 hours, about 3 hours, about 2 hours, about 1 hour. In many cases, the suitable time will be around 3 hours.

The step of conjugating the linker or effector molecule to the red blood cell results in the formation of an RBC-linker or RBC-effector conjugate. Following the formation of the RBC-linker conjugate or RBC-effector molecule conjugate, such as following the contacting step, there may be a washing step. In other words, the RBC-linker conjugate or RBC-effector molecule conjugate may be washed. Washing may remove components of the mixture that have not been conjugated, such as effector molecule or linker that is not conjugated to the RBC. The washing may remove ligase or streptavidin. Suitable washing methods will be appreciable to the skilled person, and may include washing in buffer, such as washing in PBS. The method may involve 1, 2, 3, 4 or more washes, or any number of washes such that the RBC-linker conjugate or RBC-effector molecule conjugate is substantially free from unconjugated linker or effector molecule or ligase.

Where the first contacting step involves the conjugation of an RBC to a linker, a further contacting step may be used. Such a step involves the contacting of an RBC-linker conjugate with an effector molecule. The contacting may be performed at room temperature, such as around 20° C. The contacting may be performed in the presence of a ligase. In such cases, the temperature under which the contacting is performed may be optimized according to the temperature for optional functionality of the ligase. The temperature under which the contacting is performed may be optimized for biotin-streptavidin interaction. The temperature under which the contacting is performed may be optimized for click chemistry. The contacting may be performed for a period of time suitable for the conjugation to occur. The suitable time period will be readily appreciable to the skilled person, and may involve contacting for 1 day, less than 1 day, 12 hours, or less than 12 hours, such as about 12 hours, about 11 hours, about 10 hours, about 9 hours, about 8 hours, about 7 hours, about 6 hours, about 5 hours, about 4 hours, about 3 hours, about 2 hours, about 1 hour. In many cases, the suitable time will be around 3 hours.

The step of conjugating the linker or effector molecule to the red blood cell results in the formation of an RBC-linker-effector molecule conjugate. Following the formation of the RBC-linker-effector molecule conjugate, such as following the contacting step, there may be a washing step. In other words, the RBC-linker-effector molecule conjugate may be washed. Washing may remove components of the mixture that have not been conjugated, such as effector molecule that is not conjugated to the RBC. The washing may remove ligase. Suitable washing methods will be appreciable to the skilled person, and may include washing in buffer, such as washing in PBS. The method may involve 1, 2, 3, 4 or more washes, or any number of washes such that the RBC-linker-effector molecule conjugate is substantially free from unconjugated effector molecule and/or ligase.

Some methods disclosed herein involve conjugation to a deglycosylated red blood cell. The red blood cell may be deglycosylated prior to conjugation to a linker and/or effector molecule, or after conjugation to the linker, or after conjugation of the linker to the effector molecule. Preferably, the red blood cell is deglycosylated prior to conjugation of the linker/effector molecule. In other words, prior to any conjugation step, the red blood cell may have been subject to deglycosylation. In some case, the red blood cell is deglycosylated prior to the conjugation method. That is to say that the red blood cell provided for the initial or only contacting step has been deglycosylated. In some methods, the first step of the method is to deglycosylate the red blood cell. Methods of deglycosylating red blood cells are known in the art and include enzymatic deglycosylation, such as by contacting the red blood cell with PNGaseF, EndoH, O-glycosidase or exoglycosidase (Mannosidase, neuraminidase and/or β-N-Acetylhexosaminidase). In some methods, the red blood cell is deglycosylated with a combination of O-glycosidase and exoglycosidases.

In some methods disclosed herein, a red blood cell is conjugated to a linker comprising an N-terminal biotin moiety. Such methods may be useful for conjugating biotinylated effector molecules to red blood cells, to form red blood cell-linker-effector molecule conjugates. In such embodiments, both the linker and the effector molecule may comprise a biotin moiety. In such methods it is necessary to include a further step of contacting the red blood cell-linker conjugate with a biotin binding molecule, such as streptavidin, prior to contacting the red blood cell-linker conjugate with the biotin conjugated effector molecule.

As used herein, the term “effector molecule” refers to molecules or active substances which have a certain predictable effect, whereby the effect can be chosen at the discretion of the person working the method and modified red blood cells disclosed herein. For example, if the intention is to treat cancer, the effector molecule conjugated to the red blood cell can be an antibody against said cancer, or a protein that binds to a cancer-specific receptor or the cancer cell. Examples of an effector molecule include, but are not limited to, protein, enzyme, cell-surface marker, antibody such as a monoclonal antibody, cytokine, chemokine, antibody fragment, nanobody, therapeutic agent, and combinations thereof. In another example, the effector molecules disclosed herein can be further modified by attaching functional peptides, such as but not limited to, fluorescent proteins, tagging proteins and proteins for affinity binding and the like.

By “antibody” we include a fragment or derivative thereof, or a synthetic antibody or synthetic antibody fragment. In view of today's techniques in relation to monoclonal antibody technology, antibodies can be prepared to most antigens.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799).

Fragments, such as Fab and Fab2 fragments may be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855). Antibodies or antigen binding fragments useful in the surface functionalized red blood cells disclosed herein will recognise and/or bind to, a target molecule.

That antigenic specificity is conferred by variable domains and is independent of the constant domains is known from experiments involving the bacterial expression of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the VH and VL partner domains are linked via a flexible oligopeptide (Bird et al. (1988) Science 242, 423; Huston et al. (1988) Proc. Natl. Acad. Sd. USA 85, 5879) and single domain antibodies (dAbs) comprising isolated V domains (Ward et al. (1989) Nature 341, 544). A general review of the techniques involved in the synthesis of antibody fragments which retain their specific binding sites is to be found in Winter & Milstein (1991) Nature 349, 293-299. Antibodies and fragments useful herein may be human or humanized, murine, camelid, chimeric, or from any other suitable source.

By “ScFv molecules” we mean molecules wherein the VH and VL partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and sdAb fragments are monovalent, having only one antigen combining site. Monovalent antibody fragments are particularly useful as tags, because of their small size.

In some cases, the binding molecule is a single chain antibody, or scAb. A scAb consists of covalently linked VH and VL partner domains (e.g. directly, by a peptide, or by a flexible oligopeptide) and optionally a light chain constant domain.

In some preferred embodiments the antibody is detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The antibody may be directly labelled with a detectable label or it may be indirectly labelled. For example, the antibody may be unlabelled and can be detected by another antibody which is itself labelled. Alternatively, the second antibody may have bound to it biotin and binding of labelled streptavidin to the biotin is used to indirectly label the first antibody.

In certain methods disclosed herein, the effector molecule is a nanobody. As used herein, the term “nanobody”, also known as a single-domain antibody (sdAb) or VHH, refers to an antibody fragment consisting of a single monomeric variable antibody domain. Like a whole antibody, it is able to bind selectively to a specific antigen. With a molecular weight of usually between 12 to 15 kDa, single-domain antibodies are much smaller than common antibodies (usually between 150 to 160 kDa) which are composed of two heavy protein chains and two light chains, and even smaller than Fab fragments (roughly 50 kDa, one light chain and half a heavy chain) and single-chain variable fragments (roughly 25 kDa, two variable domains, one from a light and one from a heavy chain).

Antibodies and antigen binding fragments, such as the monoclonal antibodies and nanobodies may be directed (i.e. bind to) any suitable target. For example, the antibody or antigen binding fragment may bind to EGFR or IL8, or be a T-cell/immune cell activating antibody, or an antibody against a toxin or pathogen.

Some methods disclosed herein use an OaAEP1 ligase. OaAEP1 ligases are derived from the plant species Oldenlandia affinis (O. affinis). OaAEP1 ligase is a prokaryotic enzyme from the asparaginyl endopeptidase (AEP) family of enzymes. AEP enzymes typically act as proteases, but some AEP enzymes, such as OaAEP1, have evolved ligase functionality. A preferred OaAEP1 ligase according to the methods disclosed herein is OaAEP-Cys247Ala ligase, which has a point mutation of Cys to Ala at position 247, modifies surface proteins by recognising and cleaving a carboxyl-terminal sorting signal. For most substrates of OaAEP1 ligases, the recognition signal consists of the motif —N—X₁L (Asn-X₁-Leu, where X₁ is any amino acid, preferably any amino acid selected from A, C, D, E, F, H, K, G, I, L, M, N, P, O, R, S, T, V, W or Y, most preferably G, D or P). Cleavage occurs between the asparagine (N) and adjacent residue. In some case, the ligase recognition sequence comprises sequence GG (Gly-Gly) at the N-terminus. In such cases, the ligase recognition signal may comprise the motif LPXTG (Leu-Pro-any-Thr-Gly). OaAEP1 ligase, and the OaAEP!-Cys247Ala ligase is described in WO2018/056899A1, the entire contents of which are hereby incorporated by reference. The ligase may have a sequence having at least 80% identity, at least 85% identity, at least 90% identity, at least 95% identity, at least 98% identity or 100% identity a sequence set out in the table below, preferably at least 95% identity, at least 98% identity or 100% identity.

| | |
|---|---|
| Amino acid sequence of the | ARDGDYLHLPSEVSR |
| shortest functional form of | FFRPQETNDDHGEDS |
| the activated enzyme (OaAEP1 | VGTRWAVLIAGSKGY |
| C247A) used in the examples | ANYRHQAGVCHAYQI |
| (the bold amino acid is | LKRGGLKDENIVVFM |
| the 247^{th} substitution of | YDDIAYNESNPRPGV |
| C to A): | IINSPHGSDVYAGVP |
| | KDYTGFEVNAKNFLA |
| | AILGNKSAITGGSGK |
| | VVDSGPNDHIFIYYT |
| | DHGAAGVIGMPSKPY |
| | LYADELNDALKKKHA |
| | SGTYKSLVFYLEACE |
| | SGSMFEGILPEDLNI |
| | YALTSTNTTESSWAY |
| | YCPAQENPPPPEYNV |
| | CLGDLFSVAWLEDSD |
| | VQNSWYETLNQQYHH |
| | VDKRISHASHATQYG |
| | NLKLGEEGLFVYMGS |
| | NPANDNYTSLDGNAL |
| | TPSSIVVNQ |
| An alternate form that is | ARDGDYLHLPSEVSR |
| formed during the activation | FFRPQETNDDHGEDS |
| process of the enzymes | VGTRWAVLIAGSKGY |
| (which is also functional). | ANYRHQAGVCHAYQI |
| It includes 4 additional | LKRGGLKDENIVVFM |
| amino acids due to an | YDDIAYNESNPRPGV |
| additional cleavage site | IINSPHGSDVYAGVP |
| present at the 351^{th} | KDYTGFEVNAKNFLA |
| position (underlined) | AILGNKSAITGGSGK |
| | VVDSGPNDHIFIYYT |
| | DHGAAGVIGMPSKPY |
| | LYADELNDALKKKHA |
| | SGTYKSLVFYLEACE |
| | SGSMFEGILPEDLNI |
| | YALTSTNTTESSWAY |
| | YCPAQENPPPPEYNV |
| | CLGDLFSVAWLEDSD |
| | VQNSWYETLNQQYHH |
| | VDKRISHASHATQYG |
| | NLKLGEEGLFVYMGS |
| | NPANDNYTSLDGNAL |
| | TPSSIVVNQRDAD |

As used herein, the term “sortase” refers to a group of prokaryotic enzymes which modify surface proteins by recognising and cleaving a carboxyl-terminal sorting signal. For most substrates of sortase enzymes, the recognition signal consists of the motif LPXTG (Leu-Pro-any-Thr-Gly), then a highly hydrophobic transmembrane sequence, followed by a cluster of basic residues, such as for example arginine. Cleavage occurs between the threonine (Thr) and glycine (Gly) residues, with transient attachment through the threonine (Thr) residue to the active site cysteine (Cys) residue, followed by transpeptidation that attaches the protein covalently to cell wall components. Sortases occur in almost all Gram-positive bacteria and the occasional Gram-negative bacterium (e.g. Shewanella putrefaciens) or Archaea (e.g. Methanobacterium thermoautotrophicum). Sortase is commercially available from a number of sources, such as Creative Enzyme (EXWM-4247) and Active Motif (13100). A particularly preferred sortase is the sortase A heptamutant encoded by the pet30b-7M SrtA plasmid, deposited at Addgene as #51141.

Some methods disclosed herein involve the interaction of biotin and streptavidin. Biotin is a small and stable heterocyclic compound that can be readily engineered onto proteins and peptides without changing the function or activity of that protein or peptide. A number of proteins are known to bind to biotin, including streptavidin, forming a strong non-covalent interaction. Kits and methods for conjugating proteins or peptides to biotin are well known in the art and readily appreciable to the skilled person, such as the Biotin Conjugation Kit (Type B, Abcam™, ab201796).

Effector molecules useful in the methods described herein may be modified. In particular, an effector molecule may be engineered to facilitate conjugation of the effector molecule to a red blood cell membrane protein or to a linker. In particular, an effector molecule may comprise, or may be engineered to comprise, a ligase recognition sequence, a biotin or streptavidin moiety or an azide, tetrazine, methyl tetrazine, diarylcytooctyne (DBCO) or Transcyclooctyne (TCO) moiety.

In some aspects, the effector molecule comprises a C-terminal ligase recognition sequence, preferably a C-terminus ligase recognition sequence. The specific ligase recognition sequence will depend on the ligase that is to be used to effect the conjugation. Preferably, the ligase recognition sequence is an OaAEP1 ligase recognition sequence. The ligase recognition sequence may be any ligase recognition sequence selected from: NGL, NDL, NPL, NAL, NCL, NEL, NFL, NHL, NKL, NIL, NLL, NQL, NRL, NSL, NTL, NVL, NWL, NYL, NSLD, NSLAN or NG. In preferred method, the effector molecule comprises a C-terminal NGL, NDP or NPL motif, most preferably NGL. In some methods, the effector molecule comprises an -NGL motif at the C-terminus, such as being engineered to comprise the motif at the C-terminus. In some methods, the effector molecule comprises -GG at the C-terminus, such as being engineered to comprise the motif at the N-terminus, for example where the effector molecule is engineered to comprise the sequence LPXTGG. Methods of engineering proteins and peptides to comprise additional sequences, such as an additional ligase recognition sequence/motif are well known to those of skill in the art, such as those described in Sambrook et al. Such effector molecules may be used in a “one-step” method, where the effector molecule is conjugated directly to the RBC. Such effector molecules may also be used in a “two-step” method, where the RBC is first conjugated to a linker, and subsequently the effector molecule is conjugated to the linker.

In other aspects, the effector molecule comprises a C-terminal biotin motif, preferably a biotin motif at the C-terminus. The effector molecule may have been engineered to comprise the biotin moiety at the C-terminus. Methods of engineering proteins and peptides to comprise additional sequences, such as adding a biotin moiety are well known to those of skill in the art, such as those described in Sambrook et al.

In yet other aspects, the effector molecule comprises a C-terminal azide moiety. The effector molecule may comprise a C-terminal azide molecule following conjugation of the effector molecule with Azide. Methods and kits for conjugation of proteins and peptides to Azide moieties are well known in the art, such as the NHS-Azide kit from ThermoFisher™ (Catalogue number 88902). The effector molecule may comprise a C-terminal DBCO moiety.

Effector molecules may be further engineered to facilitate functionality. For example, where the effector molecule is a nanobody or other antibody fragment, it may be appropriate to engineer the effector molecule to include one or more linker sequences to facilitate folding of effector molecule.

Effector molecules may be further engineered to include additional sequences such as tags or labels, such as to facilitate engineering, monitoring or tracking of the effector molecule. Tags or labels for proteins and peptides are well known in the art, as are methods for engineering proteins or peptides to include such sequences. For example, the effector molecule may comprise one or more of a His tag (6×His), FLAG tag, Myc tag or biotin.

Certain methods described herein involve a linker. The linker comprises a sequence of amino acids and is useful for connecting an effector molecule to a red blood cell, without conjugating the effector molecule directly to the red blood cell. Such a linker may allow connection to the red blood cell without impeding the function of the effector molecule, such as steric hindrance though proximity to the red blood cell or by causing distortion of folding of the effector molecule.

The linker may comprise or consist of the amino acid sequence:

[A]-[Y]-[B]
wherein [A] is a ligase recognition sequence, biotin, azide or DBCO moiety.
wherein [Y] is the linker body sequence; and
wherein [B] is a ligase recognition sequence.

Where [A] is a ligase recognition sequence, it has the amino acid sequence NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

Where [A] is a ligase recognition sequence, [B] has the amino acid sequence of Xaa₁GG, wherein Xaa₁ is any amino acid except G, or [B] has the sequence NG, GGG or NCL. In some aspects wherein [A] is a ligase recognition sequence, [B] has the amino acid sequence LPX₃TGG, where X₃ is any amino acid. Preferably, X₃ is E, glutamic acid, and thus the ligase recognition sequence has the amino acid sequence LPETGG.

In some aspects, [A] comprises an azide, tetrazine, methyl tetrazine or, diarylcytooctyne (DBCO) or Transcyclooctyne (TCO) moiety or a biotin moiety.

Where [A] comprises an azide, tetrazine, methyl tetrazine or, diarylcytooctyne (DBCO) or

Transcyclooctyne (TCO) moiety or a biotin moiety, [B] has the amino acid sequence NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

The linker body, [Y] may comprise any suitable sequence of amino acids. Preferably, [Y] comprises a sequence of amino acids consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. In some cases, [Y] comprises an α-helical peptide.

In some cases, [Y] comprises the sequence EAAAK (Glu-Ala-Ala-Ala-Lys). In some cases, [Y] comprises repeats of the sequence EAAAK, such as 1, 2, 3, 4, 5, 6, 7, 8, 9 or 10 repeats. Preferably, the linker comprises 1-10 or 1-5 repeats of EAAAK. Most preferably, the linker comprises 1 or 5 repeats of EAAAK.

In some cases, the linker comprises or consists of the sequence EQKLISEEDL.

In some cases, the linker comprises or consists of 29 amino acids and comprises a N terminal GL motif and an C terminal LPETGG motif. In some cases, the linker consists of the sequence of the GN20 linker, i.e. GL-GEQKLISEEDLG-LPETGG.

In some cases, the linker does not comprise a streptavidin moiety.

As used herein, the term “native red blood cell” refers to a previously untreated red blood cell. As a person skilled in the art will appreciate, a native red blood cell is one which is enucleated, with a biconcave shape. That is to say, said red blood cell has not been modified genetically (or otherwise) prior to use according to the methods disclosed herein. In particular, the red blood cell may not have been genetically modified with respect to its membrane. Thus, the membrane of the red blood cells used in the methods disclosed herein may be indistinguishable from the membrane of a red blood cell within an individual of the same species from which the red blood cell of the method disclosed herein is derived. In particular aspects, the RBC may not have been enucleated from a genetically modified erythrocyte. The RBCs described herein are intact red blood cells. The RBCs are not red blood cell derived extracellular vesicles.

It is of note that red blood cells and red blood cell extracellular vesicles are different with regard to the surface proteins they comprise or exhibit. During red blood cell extracellular vesicle biogenesis, many proteins and lipids undergo a flipping process, which makes cytoplasmic red blood cell proteins appear on the surface. Vice versa, the same flipping process results in some red blood cell surface proteins being turned to the inside of the vesicles.

Modified blood cells and compositions comprising modified red blood cells as described herein may be used in therapy, e.g. in the treatment, prevention and/or amelioration of a disease or disorder.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

The modified red blood cells described herein may be formulated for administration by a number of routes, including but not limited to, parenteral, intravenous, intra-arterial, intramuscular, intratumoural, oral and nasal.

Modified red blood cells as described herein may be used to deliver an effector molecule to a target cell. In some cases, the method is an in vitro or ex vivo method. In other cases the method is an in vivo method. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Modified red blood cells produced by the methods described herein may be associated with one or more of the following characteristics:
reduced level of glycosy residues on the red blood cell surface as compared to a native red blood cell;
at least 100,000 copies of an exogenous peptide conjugated to the exterior surface of the red blood cell, such as an effector molecule or linker peptide;
at least 100,000 molecules of biotin and optionally streptavidin conjugated to the exterior surface of the red blood cell;
the ability to bind to a target cell, such as a cancer cell, wherein the target cell expresses the ligand of the effector molecule conjugated to the red blood cell.

In an aspect, there is provided herein a deglycosylated red blood cell conjugated to an effector molecule. The effector molecule may be an antibody, antigen binding fragment or enzyme.

The methods disclosed herein provide an efficient way of surface modifying red blood cells. The methods may result in the generation of red blood cells modified with on average, at least 80,000 peptides per red blood cell, at least 90,000 peptides per red blood cell, at least 100,000 per red blood cell, or at least 10,500 peptides per red blood cell, preferably at least 100,000 peptides per red blood cell. The term “average” refers to the mean average.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Numbered Paragraphs

The following numbered paragraphs (paras) provide further statements of features and combinations of features which are contemplated in connection with the present invention

Paragraph 1. A method of surface-modifying a native red blood cell post-enucleation comprising,
a. exposing the native red blood cell obtained from a subject to an effector molecule,
b. conjugating the effector molecule to the red blood cell,
c. thereby modifying the red blood cell.

Paragraph 2. The method of paragraph 1, wherein the effector molecule has a size of at least 10 kDa.

Paragraph 3. The method of any one of the preceding paragraphs, wherein the effector molecule is selected from the group consisting of protein, enzyme, cell-surface marker, monoclonal antibody, nanobody, therapeutic agent, antibody fragment and combinations thereof.

Paragraph 4. The method of any one of paragraphs 1 to 3, wherein the conjugation step is performed using a method selected from the group consisting of one or more enzymatic reactions, biotinylation and/or streptavidin-based conjugation, or using copper-free click chemistry.

Paragraph 5. The method of paragraph 4, wherein the one or more enzymatic reactions are catalysed using one or more enzymes selected from the group consisting of butelase 1, OaAEP1 ligase, an asparaginyl peptidase, or any mutant form or variant thereof.

Paragraph 6. The method of paragraphs 4 to 5, wherein the enzymatic reaction is not catalysed by sortase.

Paragraph 7. The method of any one of the preceding paragraphs, wherein the conjugation step creates a covalent bond between the effector molecule and the native red blood cell.

Paragraph 8. The method of any one of the preceding paragraphs, wherein the native red blood cell is conjugated to the effector molecule via a linker.

Paragraph 9. The method of any one of the preceding paragraphs, wherein the native red blood cell is enucleated, with a biconcave shape.

Paragraph 10. The method of any one of paragraphs 1 to 9, wherein the enzymatic reaction is catalysed using OaAEP1 ligase, and wherein the effector molecule is a nanobody.

Paragraph 11. A modified red blood cell obtained by the method of any one of the preceding paragraphs.

Paragraph 12. A modified red blood cell comprising a post-enucleation surface-conjugated effector molecule.

Paragraph 13. The red blood cell of paragraph 12, wherein the effector molecule has a size of at least 10 kDa.

Paragraph 14. The red blood cell according to any one of paragraphs 12 to 13, wherein the effector molecule is conjugated using a method selected from the group consisting of one or more enzymatic reactions, biotinylation and/or streptavidin-based conjugation, or using copper-free click chemistry.

Paragraph 15. The red blood cell of paragraph 14, wherein the method results in a covalent bond between the effector molecule and the red blood cell.

Paragraph 16. The red blood cell of paragraph 12, wherein the enzymatic reaction is catalysed by an enzyme selected from the group consisting of butelase 1, OaAEP1 ligase, an alternative asparaginyl peptidase, or any mutant form or variant thereof.

Paragraph 17. The red blood cell according to any one of paragraphs 12 to 16, wherein the effector molecule is selected from the group consisting of protein, enzyme, cell-surface marker, monoclonal antibody, nanobody, antibody fragments, and combinations thereof.

Paragraph 18. The red blood cell of any one of paragraphs 12 to 17, wherein a linker is conjugated between the red blood cell and the effector molecule.

Paragraph 19. The red blood cell of any one of paragraphs 12 to 18, wherein the effector molecule is a monoclonal antibody.

Paragraph 20. The method of paragraphs 1 to 11, or the red blood cell of paragraphs 12 to 19, wherein the red blood cell is of human or animal origin.

Paragraph 21. The method of paragraphs 1 to 11 and 20, or the red blood cell of paragraphs 12 to 19, wherein the conjugated effector molecule exerts a therapeutic effect.

Paragraph 22. The red blood cell according to any one of paragraphs 12 to 21 for use in therapy.

Paragraph 23. Use of the modified red blood cell according to any one paragraphs 12 to 22 in the manufacture of a medicament treating a disease or disorder.

Paragraph 24. The use of paragraph 23, wherein the disease or disorder is selected from the group consisting of enzyme deficiencies, metabolic diseases, immune-related disorders, blood disorders, and cancer.

Aspects and embodiments of the present invention will now be discussed with reference to the accompanying figures. Further aspects and embodiments will be apparent to those skilled in the art. All documents mentioned in this text are incorporated herein by reference.

### EXAMPLES

### Example 1: Materials and Methods

### Purification of Red Blood Cells (RBCs)

Human whole blood was collected in citrate-phosphate-dextrose adenine buffer and stored at 4° C. until further processing. Whole blood was passed through a leuko-reduction filter to remove the majority of leukocytes. The resulting blood was washed three times using an excess of sterile phosphate-buffered saline (PBS) to remove plasma and the majority of platelets. The resulting red blood cell pellet was re-suspended in red blood cell storage buffer and stored at 4° C. for future experiments. Mouse blood was obtained via cardiac puncture into EDTA coated tubes. Blood was strained through 40 μm cell strainers to remove coagulated blood and Acrodisc® WBC (White Blood Cell) Syringe Filters to remove leukocytes. Cells were washed in an excess of PBS 3 times to remove traces of plasma and platelets. Cells were subsequently counted using a haemocytometer.

### Peptide and Nanobody Design

The peptides listed in table 1 were designed to include a C-terminal motif (-NGL) for recognition by the OaAEP1 protein ligase to facilitate ligation. Biotinylation of a single primary amine group was used to facilitate detection of peptides when required. Peptides were produced using solid phase synthesis and purified using HPLC (GL Biochem Ltd., Shanghai, China). The epidermal growth factor (EGFR) nanobody sequence was obtained from Roovers et al. (Roovers et al., 2011; DOI: 10.1002/ijc.26145) and modified to include a 6×His tag at the N terminus, and a FLAG tag and ligase-binding site at the C-terminus. Flexible linker sequences were included between each epitope tag to facilitate functionality of the nanobody as shown here: (HHHHHH-GSG-VHH-GSG-FLAG-NGL). The nanobody-encoding DNA was synthesized and inserted into pET32(a+) plasmid, following a T7 promoter, by Guangzhou IGE Biotechnology Ltd (China). The plasmid encoding OaAEP1-Cys247Ala plasmid was provided by Dr. Bin Wu, Nanyang Technology University. eGFP, recombinant human IL-8 and L-Asparaginase were obtained pre-purified from commercial sources.

TABLE 1

| | | Peptide sequence |
|---|---|---|
| | | from N to C |
| Name | Abbreviation | terminus |
| Biotinylated | TL5 | Biotin-TR-NGL |
| control | | |
| peptide | | |
| for | | |
| ligation | | |
| Linker | GN20 | GL-GEQKLISEED |
| peptide | | LG-LPETGG |
| for | | |
| ligation | | |
| of | | |
| VHH | | |
| DBCO | EK18 | DBCO-EAAAKEAA |
| peptide | | AKEAAAK-NGL |
| for | | |
| ligation | | |
| Complementary | TK3 | Biotin-TRK- |
| azide | | Azide |
| peptide | | |
| to | | |
| EK18 | | |
| Azide | GL29 | Azide-GSSGSGG |
| peptide | | EQKLISEEDLGGS |
| for | | GGSGSG-NGL |
| ligation | | |
| Complementary | GK25 | GL-GSSGSGGDYK |
| DBCO | | DDDDK-GGSGSGG |
| peptide | | K-DBCO |
| for | | |
| GL29 | | |

### Expression and Purification of Proteins

Expression and purification of recombinant nanobodies and protein ligases was conducted in an earlier part of this study. In brief, following expression in BL21 (DE3) E. coli bacteria, the bacteria were lysed and the proteins purified using a FPLC system including Ni-NITA Affinity Chromatography followed by size exclusion chromatography (SEC). The enzyme OaAEP1 was activated by subjecting purified inactive enzyme to overnight incubation at RT in acetate buffer at pH 3.7.

Conjugation of Red Blood Cells with Peptides, Nanobodies or Monoclonal Antibodies Using OaAEP1 Ligase

For ligation, 1×10⁷ red blood cells were incubated with 500 μM peptide or VHH and 0.26 mg/ml ligase in PBS buffer pH 7, in a total volume of 20 μl, at room temperature for 3 hours with gentle agitation (30 rpm) on an end-over-end shaker.

For nanobody ligation, a two-step method was utilized. First, a linker peptide (containing both N- and C-terminal motifs for the enzyme) was ligated, followed by two washes with PBS. The linker peptide-ligated red blood cells were then incubated with 500 μM VHH and 0.26 mg/ml ligase in the same condition as for the peptide ligation. After the ligation, red blood cells were washed using centrifugation at 800×g for 5 minutes at 4° C.

For the conjugation of biotinylated monoclonal antibodies, red blood cells were ligated with a biotinylated peptide. Following thorough washing, the enzymatically biotinylated red blood cells were incubated with recombinant Streptavidin protein (Abcam) at a final concentration of 0.04 μg/μl of Streptavidin at 4° C. for 30 minutes. Following another wash, these red blood cells were incubated with biotinylated monoclonal antibodies. These antibodies were either obtained commercially, or biotinylated in-house via a Biotin Conjugation Kit (Type B, Abcam). Free (that is unbound) antibody was washed off using a final washing step.

For the conjugation of DBCO-conjugated peptides/antibodies using strain-promoted alkyne-azide cycloaddition (SPAAC) or copper free click chemistry, DBCO-conjugated linker peptides were first ligated on the red blood cells. These red blood cells were then incubated with azide-conjugated molecules at room temperature for 3 to 12 hours to allow stable conjugation via click chemistry. Azide was conjugated on proteins using an Azide-NHS kit. For monoclonal antibodies, site specifically modified antibodies with azide conjugated to the glycan groups on the Fc domain were obtained commercially.

### Western Blot Analysis

Ligated red blood cells were first treated with ACK (Ammonium Chloride, Potassium) lysis buffer in the presence of a protease inhibitor (Biotool) to obtain red blood cell membrane ghosts (decreases intracellular haemoglobin content). The membranes were pelleted by centrifugation at 21,000×g for 2 hrs. Red blood cell membrane pellets were incubated with RIPA buffer supplemented with protease inhibitors (Biotool) for 15 minutes on ice. Proteins were quantified using a Pierce BCA protein assay kit and haemoglobin using a Nanodrop reader (absorbance at 420 nm and 586 nm).

The lysates were treated with Laemmli buffer and incubated at 95° C. for 5 minutes to denature proteins. Proteins were separated on either a 10% or 12% polyacrylamide gel and transferred to a polyvinylidene difluoride (PVDF) membrane (Immobilon-P). PM5100 ExcelBand 3-color protein ladder (SmoBio, Taiwan) was loaded as a marker to estimate sizes. Membranes were blocked using 5% milk (Difco Skim Milk) in Tris buffered saline containing 0.1% Tween-20 (TBST) for 1 hour followed by incubation with primary antibodies overnight at 4° C.: rabbit anti-FLAG (Sigma, Cat #F3165, dilution 1:3000). The blot was washed 3 times with TBST then incubated with horse radish peroxidase (HRP)-conjugated anti-rabbit secondary antibody (Vector, dilution 1:5000) for 1 hour at room temperature. For detection of biotinylated peptides, the blot was incubated directly with Pierce high sensitivity streptavidin-HRP (Thermo Fisher, dilution 1:5000). The blot was imaged using a Bio-Rad Chemidoc gel documentation system.

### Flow Cytometry (FACS) Analysis

Cells were washed 2× with PBS, re-suspended in 100 μl FACS buffer (PBS with 0.5% foetal bovine serum). For surface protein analysis, the cells were incubated with 2 μl fluorescent-conjugated antibody for 30 minutes on ice, in the dark, and washed twice with 1 ml FACS buffer. FACS analysis of RBCs was performed CytoFLEX-S or CytoFLEX LX cytometers (Beckman Coulter) or S1000Ex Flow Cytometer (Stratedigm). The resulting FCS files were analysed using Flowjo V10 (Flowjo, USA). The cells were first gated by FSC-A versus SSC-A to identify individual cell populations, excluding debris and dead cells. Single cells were then gated by FSC-width versus FSC-height, excluding doublets and aggregates. The fluorescent-positive population of beads or cells were subsequently gated by targeted fluorescent channels, such as FITC for AF488 or CFSE, APC for AF647 and ECD for mCherry.

### L-Asparaginase Assay

Asparagine-dependent Sup-B15 cells were cultured in IMDM media supplemented with 20% iFBS and 0.05 mM 2-mercaptoethanol. RBCs were conjugated with biotinylated L-Asparaginase as described above. Unconjugated biotinylated L-asparaginase or RBCs with or without L-Asparaginase conjugation were co-cultured with Sup-B15 cells at a 1:5 ratio (RBCs to Sup-B15 cells). 4 days after culture, 10 μL CCK8 reagent was added to each well and the plate was incubated at 37° C. for 1 hour. The absorbance was subsequently measured at 450 nm using a plate reader.

### Immunofluorescent Imaging

Red blood cells (10⁵) were washed twice in cold PBS containing 1% BSA. The volume was made up to 100 μl. Slides and pre-wet filters were prepared and the sample was pipetted into the well of each cytospin funnel. The slides were spun at 800×g for 3 minutes. The filters were removed from the slides without contacting the immobilised red blood cells on the slides. The cells were stained with the respective antibodies for 30 minutes in the dark and washed three times using BSA-PBS. The slides were covered and imaged using a Leica Thunder microscope. Imaging was conducted in a blinded manner, with images being acquired randomly, followed by blinded co-localization analysis using Coloc 2 (ImageJ).

### In Vivo Half-Life Analysis

Surface modified (biotin peptide ligated) or unmodified red blood cells were labelled with CFSE at 37° C. for 20 minutes, washed twice and injected into mice via the tail vein. Blood was collected through the submandibular vein using a lancet (via cheek bleed) at regular intervals. Whole blood was spun down, counted and 50 million cells were taken for staining with Streptavidin-AF647 at a concentration of 0.2 mg/ml. Cells were washed twice after 1 hour of staining and analysed by flow cytometry. CFSE was used to distinguish injected red blood cells from endogenous red blood cells and Streptavidin-AF647 was used to monitor the stability of the ligated peptide in vivo over time.

### Data Analysis

GraphPad Prism 8 was used to perform statistical analyses of the data and generate graphs. A P-value <0.05 was considered to be significant. Figures of experimental outlines were created with BioRender.com and/or Adobe illustrator. Western blot analysis, co-localization analysis and mean fluorescence per unit cellular area were determined using FIJI. FACS plots were generated using FlowJo V10.

### Example 2: Results

OaAEP1 Cys247Ala can be Used to Covalently Ligate Peptides on the Human Red Blood Cells (hRBC) Surface

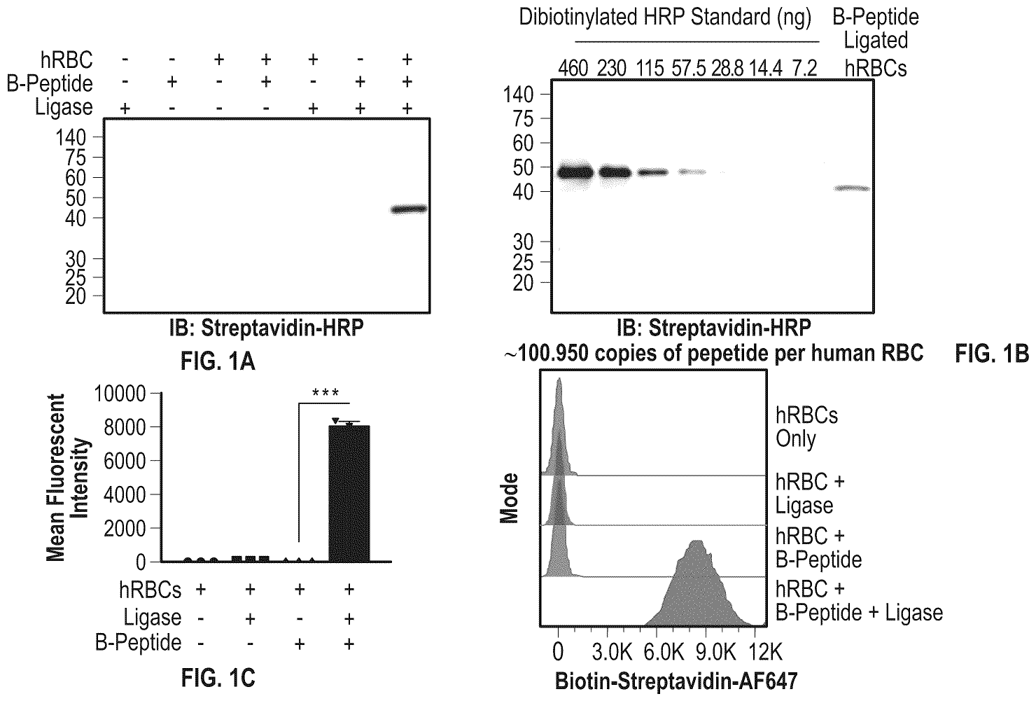

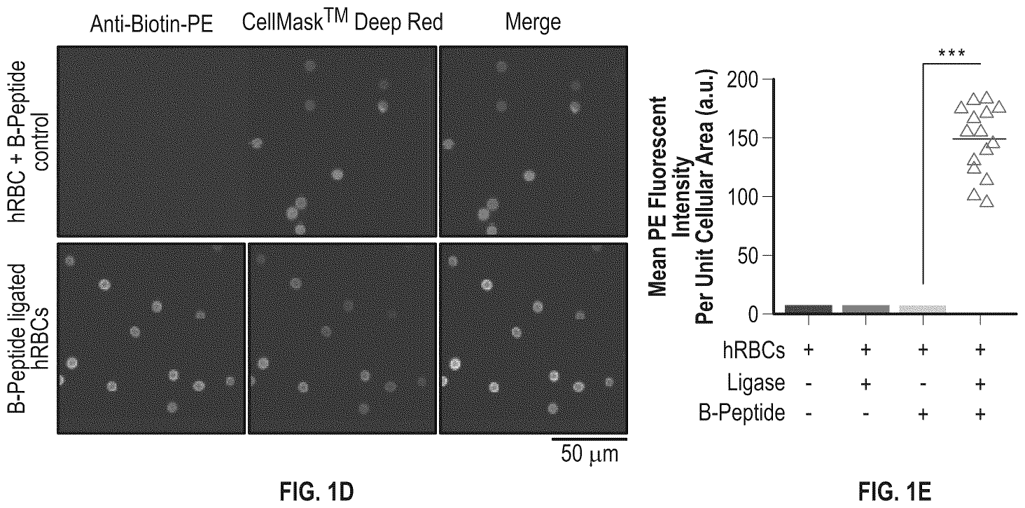

Protein ligases such as OaAEP1 or Sortase can be used to catalyse the covalent conjugation of specially designed peptides onto RBCs. To test the efficiency of OaAEP1 protein ligase-mediated conjugation of peptides on human red blood cells, a biotinylated peptide (B-Peptide/B-TL5) was designed with a ligase-recognition site for conjugation onto red blood cells. Following ligation, the resulting red blood cells were analysed by western blot for the presence of biotinylated proteins. The western blot of red blood cells ligated with a biotinylated peptide (B-TL5) revealed a clear band of biotinylated protein of about 40 kDa that was not observed in any of the controls (FIG. 1A, lanes 1 to 6). The data demonstrated that this enzymatic approach can be used to conjugate peptides and/or covalently introduce functional groups onto red blood cell membrane proteins (FIG. 1A). To further verify the efficiency of ligation, we sought to determine the copy number of peptides conjugated per human red blood cell (hRBC). Quantification of human red blood cell labelling was carried out with a monobiotinylated peptide (B-TL5) by immunoblotting with streptavidin-HRP. Dibiotinylated HRP was used as a reference for quantification. Molecular weights (kDa) of protein markers are shown on the left of each blot. Subsequent analysis revealed that on average, a single human red blood cell was conjugated with over 100,000 peptides on average (FIG. 1B). This result was further verified using flow cytometry. Biotinylated-peptide-ligated or control human red blood cells were stained with Streptavidin-AF647 and analysed by flow cytometry. Only the human red blood cells ligated with B-TL5 in the presence of the enzyme produced a significant shift in population, confirming the presence of the B-TL5 peptide on the surface of human red blood cells (FIG. 1C). Ligated and unligated human red blood cells were also observed using immunofluorescence imaging, showing the ligated peptide (stained green using a PE conjugated anti-biotin antibody) co-localized on the human red blood cell membrane (stained using CellMask Deep Red Plasma membrane Stain; FIG. 1D). The mean fluorescence from the PE-biotin staining was quantified per unit cellular area (using the CellMask staining as a mask) for roughly about 100 cells per condition for biotinylated peptide ligated human red blood cells and unligated human red blood cells and presented in FIG. 1E. Moreover, the extent of co-localization of CellMask and biotin was quantified more accurately using co-localization analysis and the Pearson's R value was found to be 0.96, confirming strong co-localization between the two signals only in the presence of successful enzymatic ligation.

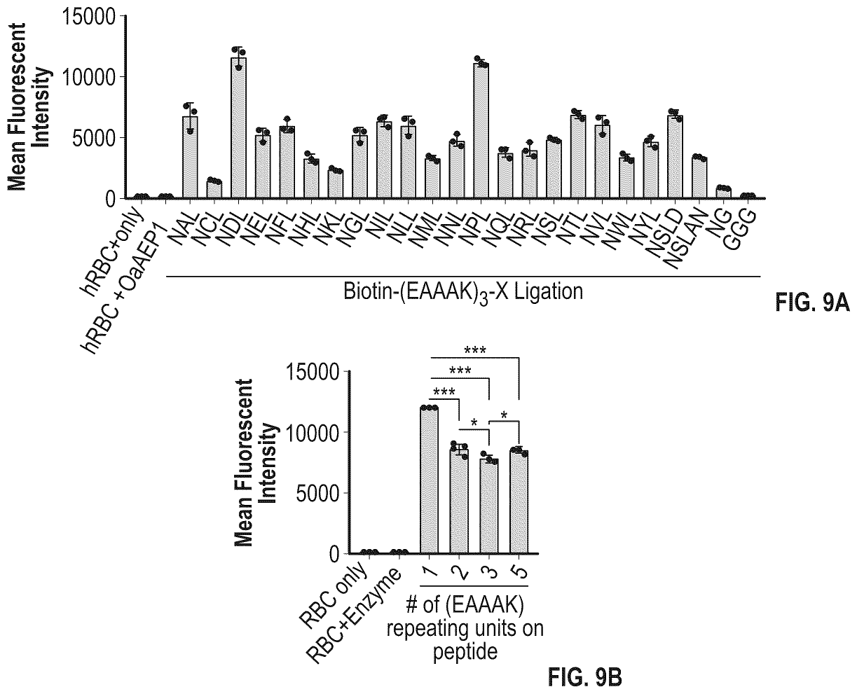

We also investigated how the peptide sequence affected ligation yield by screening the ligation efficiency of (EAAAK)3-X peptides with varying sequences asX, the C-terminal recognition motif. Interestingly we discovered that RBC ligation was functional with a diverse range of C-terminal sequences (FIG. 9A). The optimal ligation yield obtained when X was NDL or NPL, which resulted in significantly higher ligation yields.

The effect of peptide length was also assessed using an alpha helical peptide with varying numbers of (EAAAK) repeating units. Overall peptides of length from 8 amino acids up until 28 amino acids (1-5 EAAAK repeats) displayed efficient ligation. The shortest peptides tested (1 EAAAK repeat, 8 amino acids long) showed significantly higher yield than peptides with longer sequences. Increasing the number of EAAAK repeats resulted in small decreases in ligation yield from 13 to 18 amino acids (2 to 4 EAAAK repeats) (FIG. 9B). Surprisingly, the peptide with 28 amino acids (5 EAAAK repeats) showed a slight increase in ligation yield compared to the 18 amino acid peptide.

A Two-Step Method can be Used to Covalently Ligate Single Domain Antibodies on the hRBC Surface

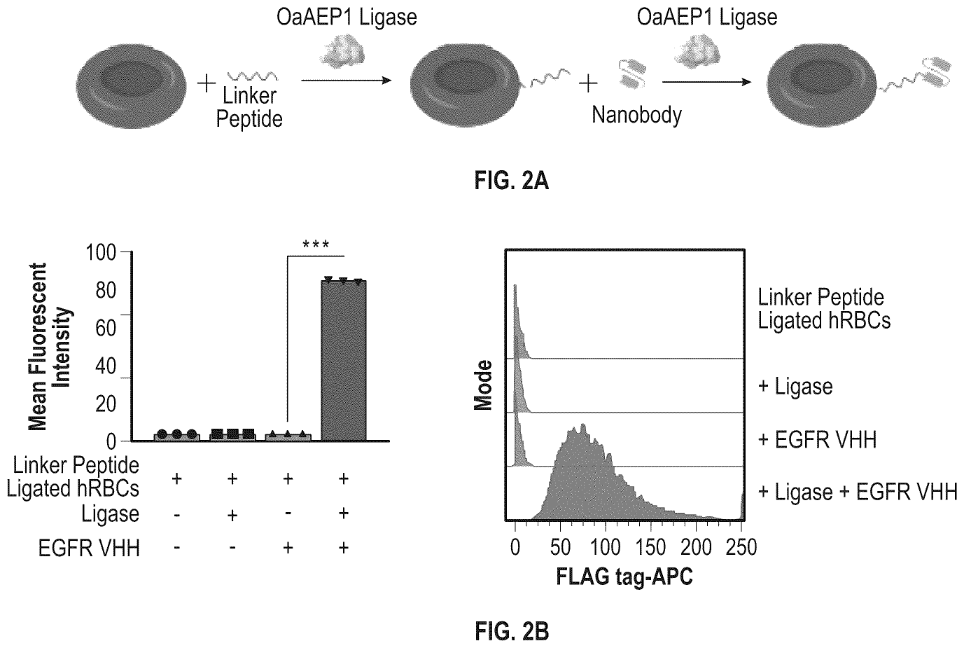

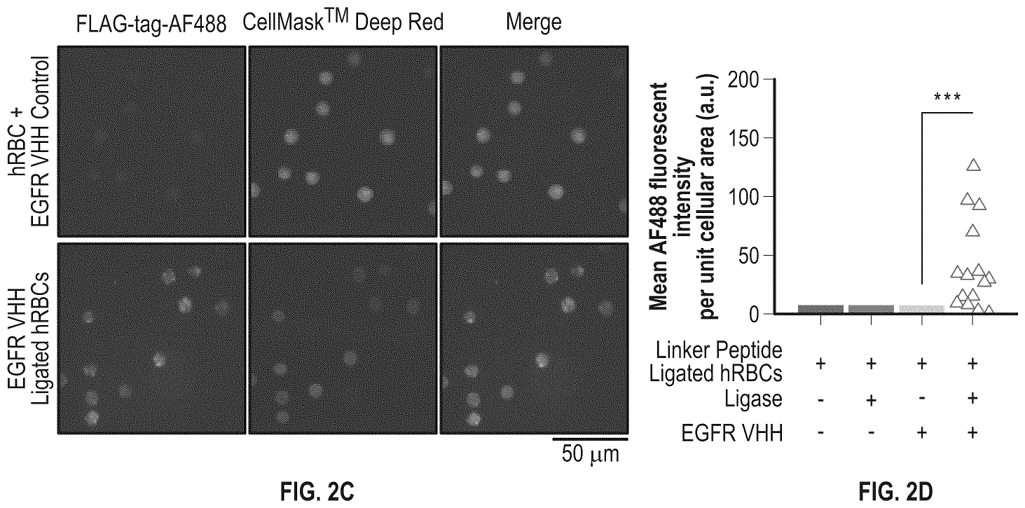

A two-step method can be used to covalently ligate single domain antibodies on the human red blood cell surface. This data also revealed that the direct ligation of significantly larger proteins, such as, but not limited to, single domain antibodies (VHHs) on the human red blood cell surface was not possible, most likely due to the larger and more complex structure. As such, a two-step method was devised for the fully enzyme-mediated conjugation of such proteins. In the first step, human red blood cells were conjugated with a linker peptide, GN20, using OaAEP1 ligase and in the second step, the linker peptide was conjugated with camelid-derived single domain antibodies (about 15 to 30 kDa) using the same enzyme (FIG. 2A). As the result, an anti-EGFR single domain antibody with FLAG tag was efficiently ligated on the human red blood cell surface using this method, as shown using flow cytometry in FIG. 2B. This data clearly shows that 100% of human red blood cells are ligated with single domain antibodies.

Immunofluorescent imaging also supported this, confirming that the two-step method was functional, albeit less efficient than peptide conjugation (FIG. 2C, showing that the FLAG tag of VHHEGFR (stained green using an anti-FLAG-AF488 antibody) co-localized on the human red blood cell membrane (stained red using CellMask™ Deep Red Plasma membrane Stain). Co-localization of CellMask and VHH was also quantified as illustrated in FIG. 2D, represented as the mean AF488 signal per unit cellular area for unligated and control human red blood cells. The Pearson's R value for the VHH-ligated human red blood cells was found to be 0.51, verifying the lower degree of VHH co-localization with the cell membrane as compared to peptide ligation.

Design of Linker Peptides for a Two-Step Method can be Used to Covalently Ligate Single Domain Antibodies on the hRBC Surface

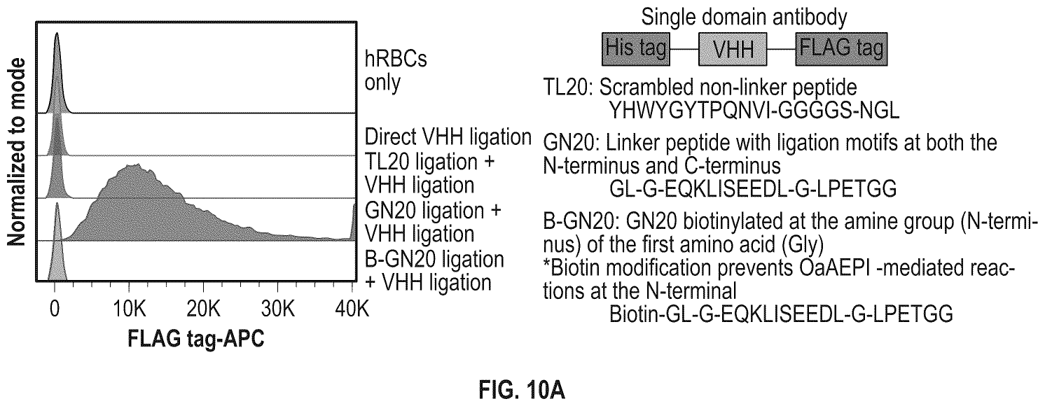

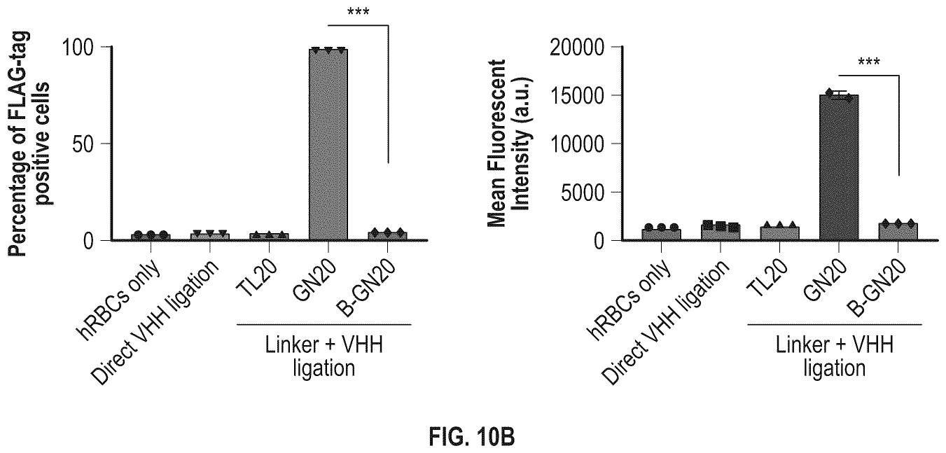

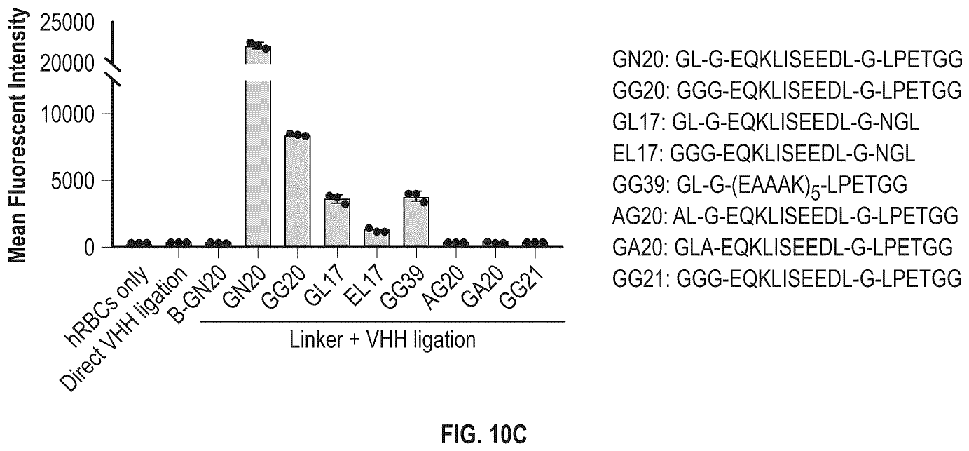

We additionally verified the efficiency of the 2-step ligation method, comparing the relative efficiency of a number of different linker peptides, each possessing enzyme recognition motifs on both their N- and C-termini. Following linker peptide ligation, the RBCs were conjugated with a single domain antibody against EGFR (EGFR VHH). The FLAG-tagged single domain antibody was subsequently detected on the RBC surface using an anti-FLAG tag antibody. Initial flow cytometric analysis revealed that direct ligation of the nanobody on RBCs did not result in any noticeable ligation (FIG. 10A-B). However, in the presence of specially designed linker peptides with specific recognition motifs at both termini (GN20), we were able to observe efficient EGFR VHH conjugation of 100% of hRBCs. Of note, scrambled non-linker peptides of similar length (TL20) or the addition of biotin on the N-terminal of GN20 (B-GN20) abrogated the ligation, indicating the efficiency of 2-step linker peptide method for conjugation (FIG. 10A-B).

Further analysis comparing different variations of N- and C-terminal ligation motifs revealed that the combination used in GN20 peptide gave the optimal yield, while the peptides that we used previously for RBCEV conjugation, GG20, EL17 and GL17, showed markedly lower ligation efficiency (FIG. 10C). The newly designed GN20 peptide gave the best ligation yield, resulting in 100% conjugation of RBCs and an approximately 3-fold increase in copy number over the next best peptide, GG20 (FIG. 10C). The higher copy number obtained via GN20 is attributed to the optimized ligation motifs on GN20 that facilitates more efficient ligation at the N-terminal to the VHH in the second step of ligation (FIG. 10B). Our data also revealed a limited variation in the N- and C-terminal motifs for 2-step ligation, wherein changing the N-terminal motif of GN20 from GLG- to GLA- or ALG- completely abrogated the ligation reaction (FIG. 10C).

Furthermore, the addition of an additional G to the C-terminal motif -LPETGG (resulting in -LGETGGG) also completely inhibited the 2-step ligation. However, we were able to demonstrate that the internal sequence and the length of the GN20 peptide could be modified without completely impairing the 2-step ligation as demonstrated by the efficient ligation of hRBCs with VHHs using the GG39 linker peptide (FIG. 10C).

### Example 2: Deglycosylation of RBCs Facilitates the Ligation of Proteins onto RBCs

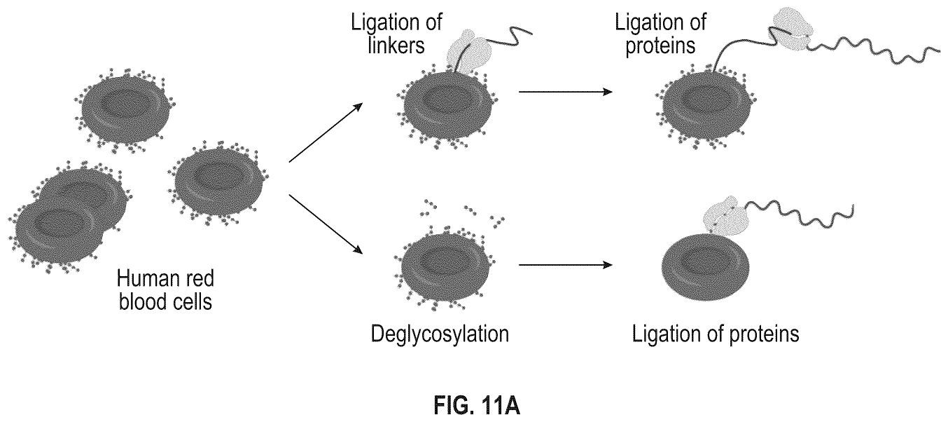

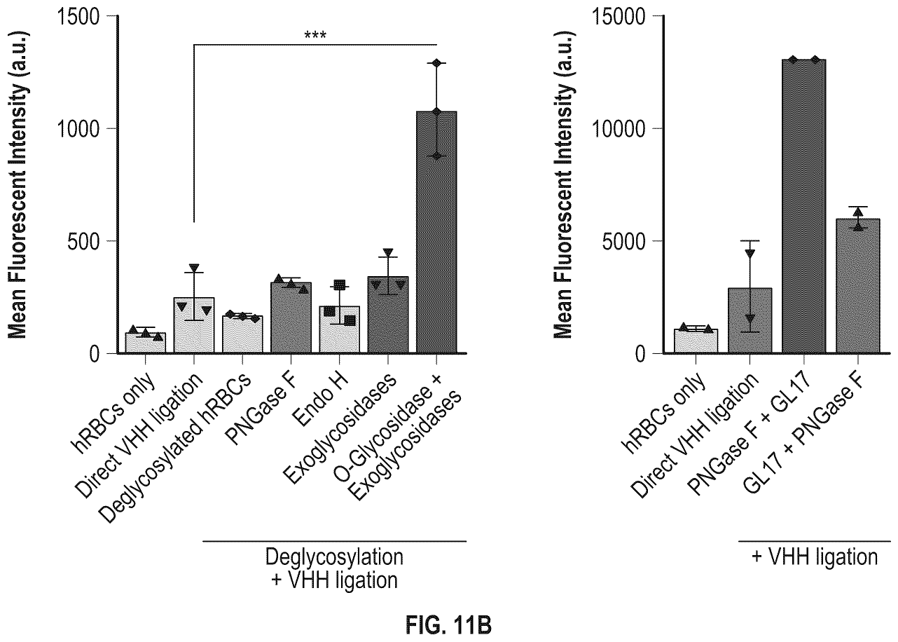

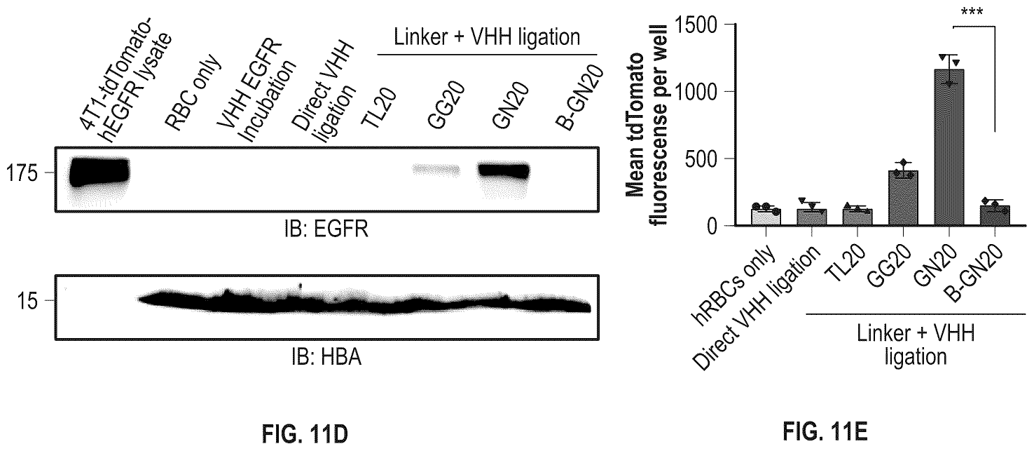

We also compared the effect of deglycosylating RBCs prior to ligation of proteins (FIG. 11A). Of note, we demonstrate that deglycosylation itself has no effect on the basal fluorescence of RBCs (FIG. 11B). However, upon subsequent ligation of a protein (EGFR VHH), we were able to detect increased ligation efficiency, with the most appreciable increase seen following O-glycan removal (FIG. 11B). Significantly, we were able to demonstrate that the deglycosylation strategy was synergistic with the 2-step ligation method outline above by carrying out both deglycosylation and linker peptide-mediated ligation prior to EGFR VHH ligation (FIG. 11C). Interestingly, deglycosylating with PNGase F and ligating with the GL17 linker peptide resulted in an appreciable increase in EGFR VHH ligation yield which was much higher that the yield obtained with either condition alone. Notably, we see a greater increase in ligation yield when deglycosylation was carried out prior to linker peptide ligation and not the other way around, suggesting that the deglycosylation process was able to facilitate the more efficient conjugation of the linker peptide and subsequently increase EGFR VHH conjugation (FIG. 11C).

### Example 4: RBCs Ligated with EGFR-Binding Single Domain Antibody can Attach to EGFR-Positive Metastatic Breast Cancer Cells

We also verified the functionality of EGFR VHH-conjugated RBCs by briefly incubating either control RBCs or GN20-linked EGFR VHH ligated RBCs with 4T1 cells expressing tdTomato and human EGFR (hEGFR) for 10 minutes. The RBCs were subsequently separated from the 4T1-tdTomato-hEGFR cells, lysed and the efficiency of cancer cell pulldown was analysed by western blotting for EGFR. As shown in FIG. 11D, only the GN20 linker peptide was able to result in sufficient EGFR VHH conjugation to provide an appreciable pulldown of cancer cells, confirming the ability of these engineered RBCs ligated with single domain antibodies to bind to cancer cells expressing the corresponding receptor. HBA is used as an internal control for RBCs, demonstrating that equal quantities of RBCs were used for pulldown in each condition. We also verified this data by reading the tdTomato fluorescence of each pulldown sample using a plate reader (FIG. 11E). 4T1 cells are metastatic breast cancer cells that usually disseminate from the primary tumour to distanced sites through the circulation and form metastases at high frequency. Although 4T1 cells express EGFR naturally, the human homologue of EGFR is expressed on 4T1 cells in this study to mimic human cancer cells. EGFR is a common receptor in human cancer including breast cancer. Hence, the binding of EGFR-VHH coated RBCs to 4T1 cells suggests that such RBCs could be transfused into patients with metastatic cancer to recognize circulating EGFR-positive tumour cells. The RBC-tumour cell duplex could then be removed or eliminated to reduce the metastatic rate.

### Example 5: Conjugation of Larger, More Complex Proteins onto the Human Red Blood Cell (hRBC) Surface Via a Streptavidin Linker

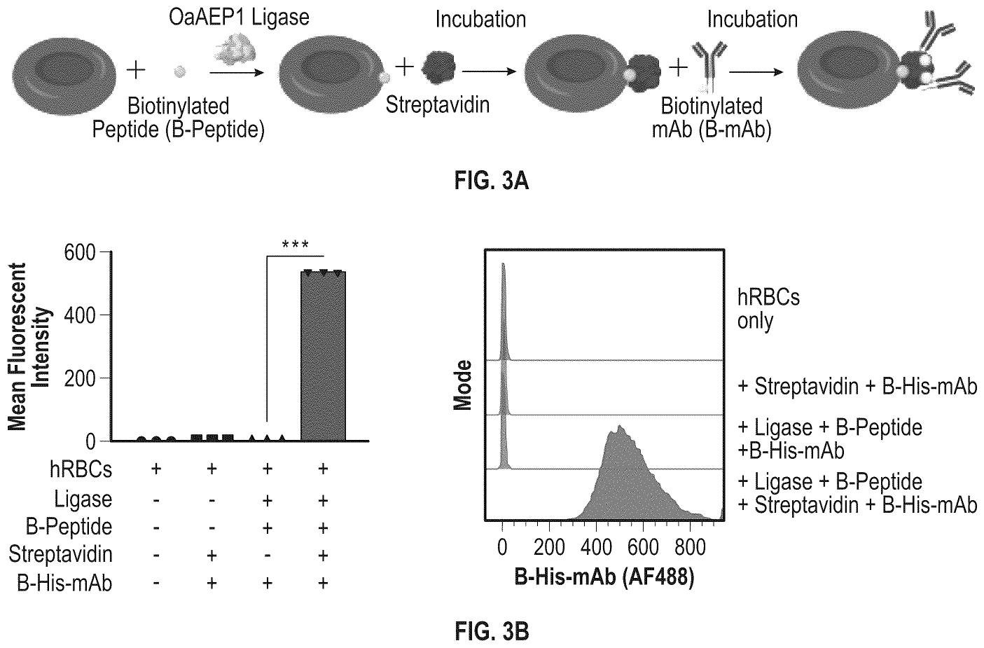

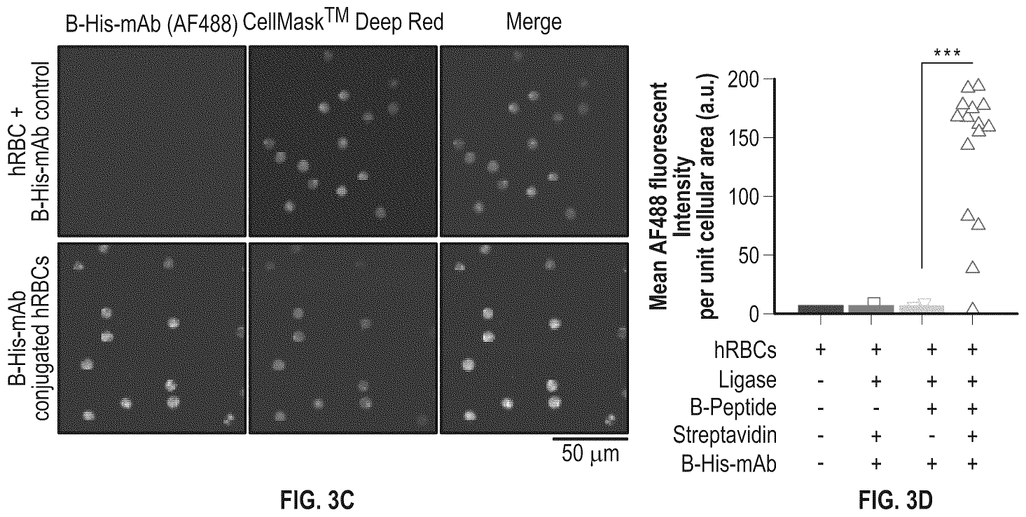

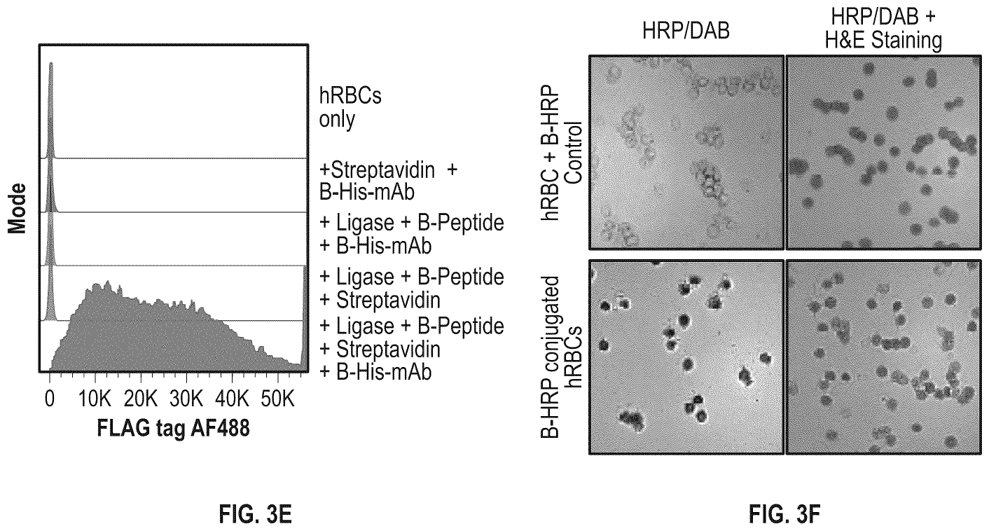

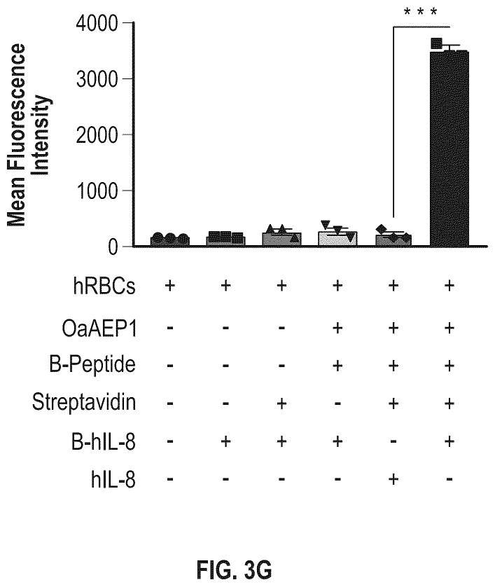

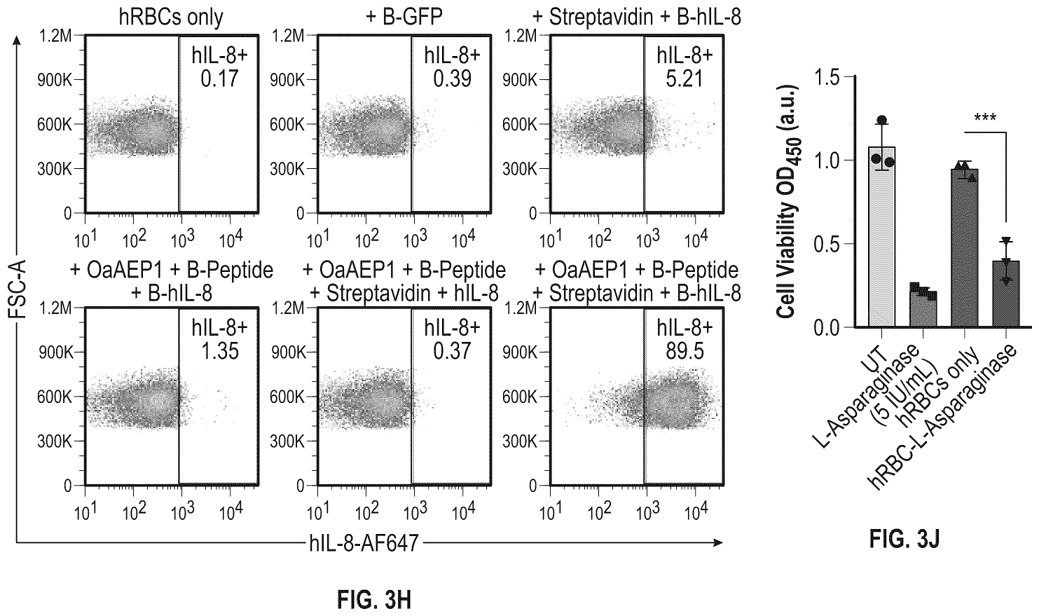

While a two-step linker peptide conjugation method for relatively small to medium sized proteins onto human red blood cells had been established, it was further sought to find a method to immobilize larger proteins such as monoclonal antibodies (mAbs) and enzymes on the human red blood cells surface. To this end, a modular streptavidin-mediated conjugation approach was established based on simple incubation of biotinylated peptide ligated human red blood cells sequentially with streptavidin and a biotinylated protein of choice as outlined in FIG. 3A. Despite being an affinity interaction, the streptavidin method was demonstrated to be very stable and the four binding sites on each streptavidin molecule enhanced the copy number of the biotinylated protein of interest per red blood cell. Biotinylated anti-his tag monoclonal antibody-conjugated human red blood cells were analysed using flow cytometry, confirming the presence of monoclonal antibodies on the human red blood cell surface, as visualized by the large shift in fluorescence of the entire population following staining with a secondary fluorescent antibody (FIG. 3B). Immunofluorescence imaging, showing a biotinylated rabbit monoclonal antibody (stained green using a donkey anti-rabbit AF488 antibody) co-localized on the human red blood cell membrane (stained red using CellMask™ deep red plasma membrane stain) was also used to demonstrate successful conjugation of the monoclonal antibody on the human red blood cells at high efficiency (FIG. 3C). Co-localization analysis revealed a Pearson's R value of 0.86 for monoclonal antibody conjugated human red blood cells, which is significantly higher than the two-step linker peptide method used previously for VHH conjugation indicating the higher efficiency of this approach. FIG. 3D also presents the mean fluorescence per unit cellular area (with reference to cell mask) in the AF488 channel for each condition. Monoclonal antibody conjugated human red blood cells were shown to be functional, being capable of pulling down target antigens from solution as demonstrated by the pulldown of his-tagged protein (also containing a FLAG tag for detection) by biotinylated anti-his-tag antibody conjugated human red blood cells (FIG. 3E). Target antigens were detected on the surface of human red blood cells using a FLAG-tag antibody. To further demonstrate the versatility of this approach, biotinylated HRP was also conjugated to the surface of red blood cells. Red blood cells were bleached of endogenous peroxidase and then conjugated with biotinylated HRP followed by incubation with DAB chromagen (3,3′-Diaminobenzidine), followed by H&E staining. Horseradish Peroxidase (HRP) activity was measured by the formation of the characteristic brown precipitate (FIG. 3F). This also demonstrates that enzymes remain functional on the RBC surface following conjugation.

We further demonstrate that the RBC conjugation approach is translatable to a range of different proteins of varying size and function in FIGS. 3G-H (human IL-8 conjugation detected using an anti-IL-8 antibody) and FIG. 31 (L-Asparaginase conjugation resulting in efficient viability suppression of Asparagine-dependent Sup-B15 cells in a co-culture with engineered RBCs). Of note, the conjugated proteins are shown to retain their function as demonstrated by the endogenous fluorescence of GFP, and the ability of L-Asparaginase conjugated hRBCs to decrease Sup-B15 cell viability and proliferation.

### Fully Bio-Orthogonal, Covalent, Efficient Conjugation of Large Molecules on Human Red Blood Cells Via Enzymatic Ligation and Click Chemistry.

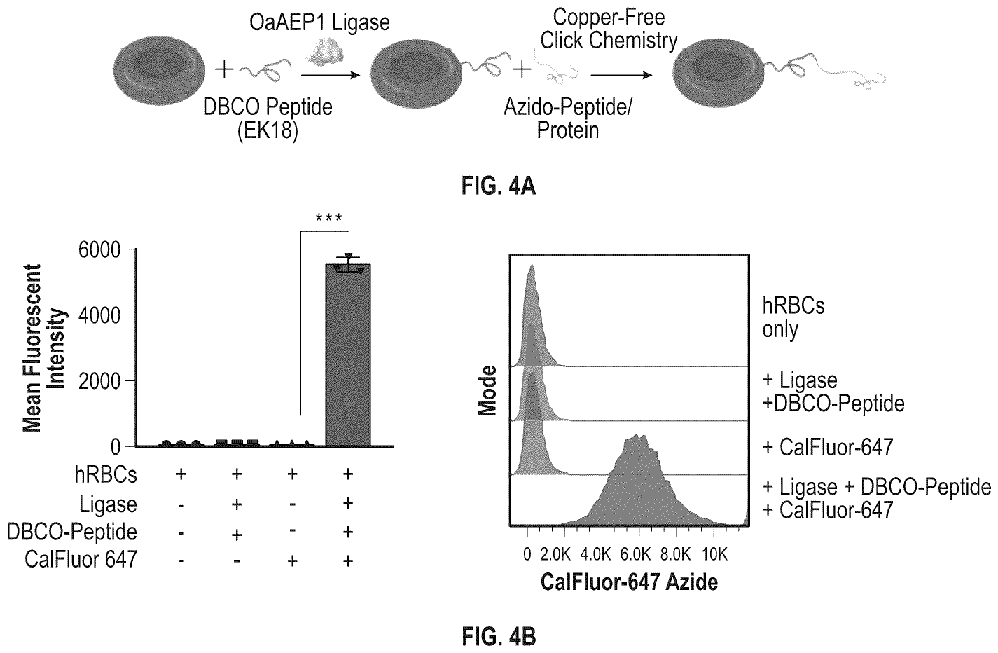

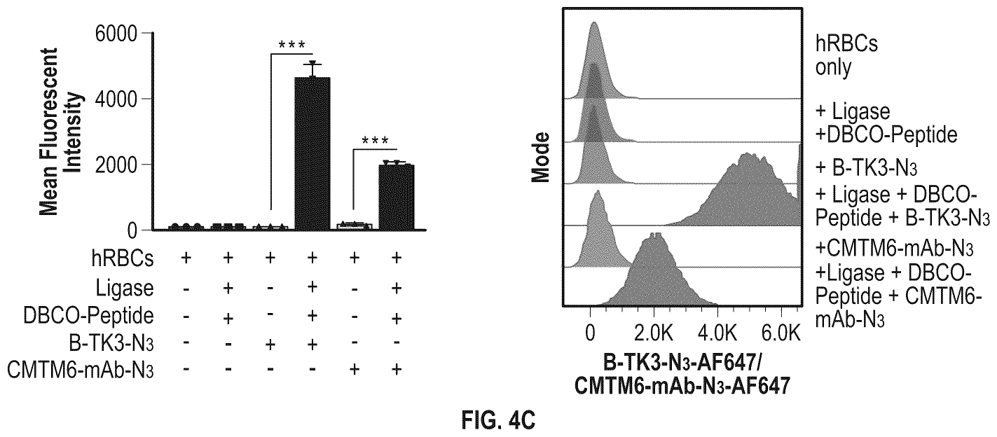

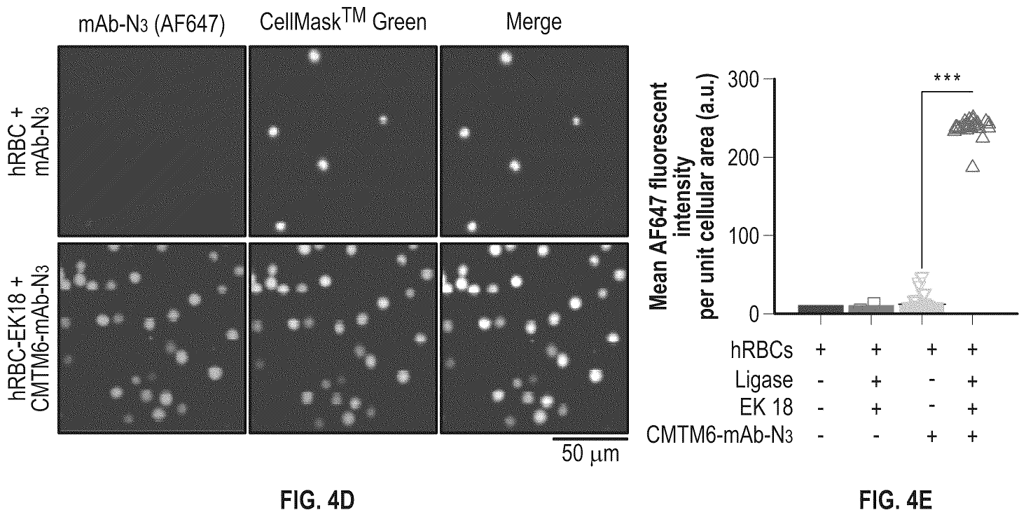

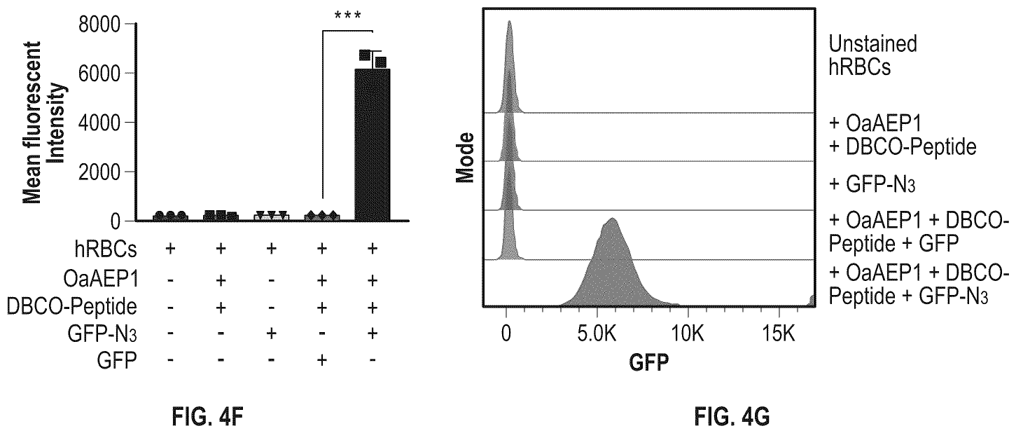

While the streptavidin method demonstrated the ability to efficiently conjugate human red blood cells with large molecules, the streptavidin molecule in itself possesses immunogenic properties, given its bacterial origin that disfavors its use in certain clinical applications. As such, we utilized a combination of copper-free click chemistry and enzymatic ligation to demonstrate completely bio-orthogonal conjugation of any azide/DBCO-tagged molecule onto the human red blood cell surface as illustrated in FIG. 4A. The use of the first enzymatic step allows the biocompatible and covalent introduction of the first reactive group (in this case, a peptide tagged with DBCO) onto the human red blood cell membrane without the need for any harsh chemical modifications. Secondly, the use of copper-free click chemistry/SPAAC (strain-promoted alkyne-azide cycloaddition) allows the subsequent conjugation of a second larger functional molecule tagged with the complementary reactive functional group (in this case an azido-antibody) onto the human red blood cell surface. The entire process is fully biocompatible, utilizes no immunogenic molecules and results in fully covalent conjugation of the molecule of interest. To demonstrate successful click chemistry, hRBCs were conjugated with a DBCO-tagged peptide (EK18), followed by incubation with CalFluor 647 azide, a fluorogenic azide probe that only emits fluorescence upon activation via click chemistry. FIG. 4B shows the mean fluorescence of CalFluor 647 that was incubated with DBCO-peptide ligated and unligated hRBCs, clearly showing the ability of the EK18 peptide ligated hRBCs to undergo copper-free click chemistry reactions. More importantly, 100% of hRBCs were positive for the CalFluor 647 fluorescence, indicating efficient conjugation of all hRBCs. FIG. 4C shows the successful conjugation of either an azido-peptide (TK3) or an azido-monoclonal antibody onto hRBCs with all controls, demonstrating that enzymatic ligation of the EK18 peptide is a prerequisite for successful click chemistry. The data also demonstrates that the monoclonal antibody (mAb) conjugation is slightly less efficient that the peptide conjugation, possible owing to the larger size of the monoclonal antibody and the lower concentration used for the conjugation step. However, it is noteworthy that >95% of RBCs were positive for both the peptide and monoclonal antibody, despite the variation in copy number, as indicated by the shift of the entire population in the FACS histogram (FIG. 4C). Efficient monoclonal antibody conjugation via click chemistry is also demonstrated via immunofluorescent imaging, where intense fluorescence is detected on the cell surface via a secondary antibody only on the human red blood cells that have undergone successful click chemistry (FIG. 4D). FIG. 4E further summarizes this data as mean fluorescence per unit cellular area for each condition. To demonstrate the translatability of this approach to other molecules, we also demonstrated that other proteins of different size such as GFP can also be conjugated successfully onto RBCs using this method (FIG. 4F-G).

### Example 6: The Human Red Blood Cells (hRBCs) Ligation Approach is Translatable to Mouse Red Blood Cells (mRBCs)

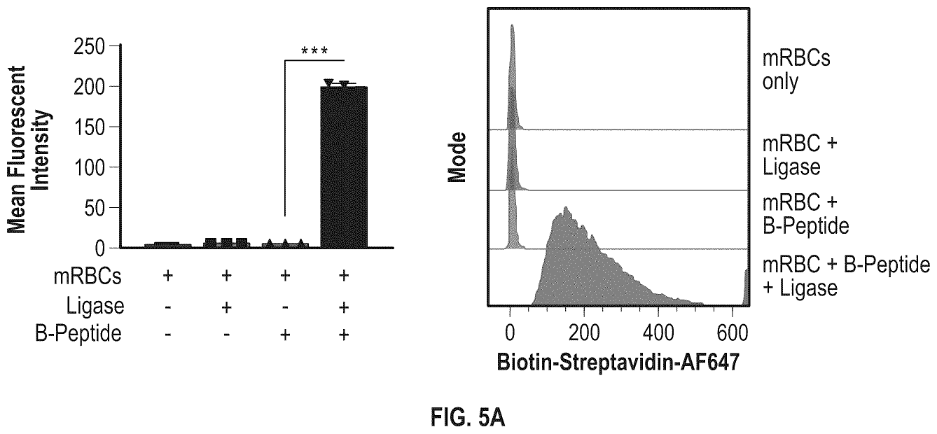

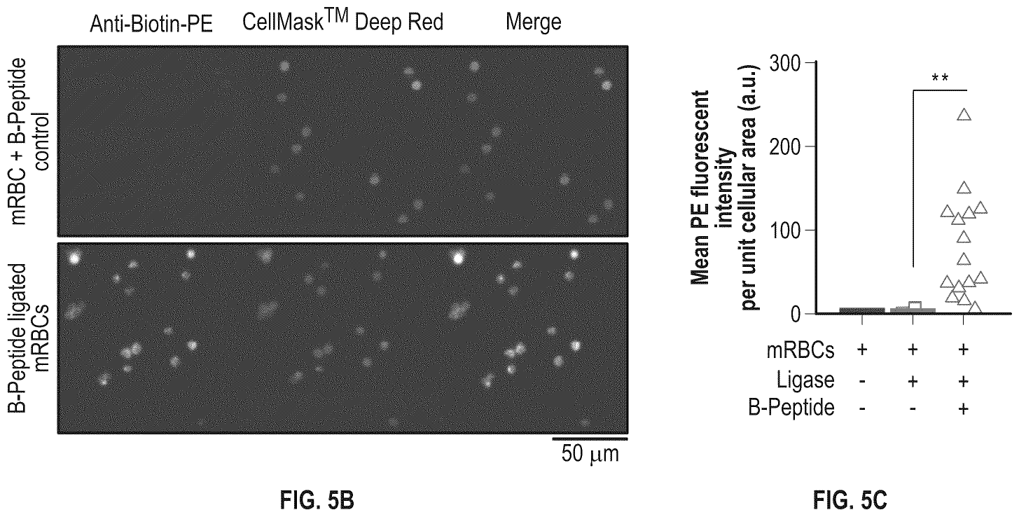

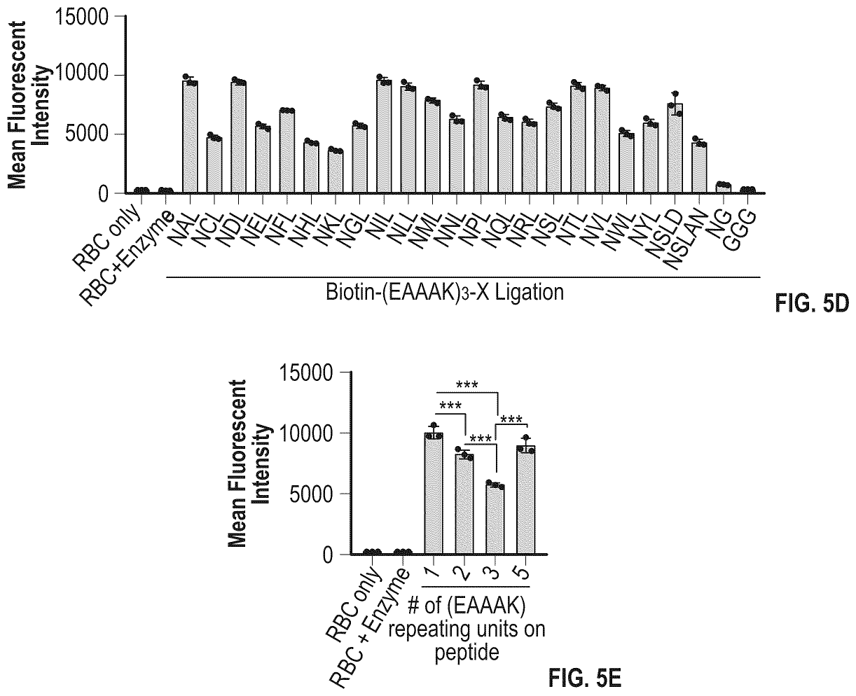

Thus far, all experiments were conducted on human red blood cells (hRBCs). It was sought to verify if mouse red blood cells (mRBCs) could also be modified in a similar manner. Flow cytometry (FIG. 5A) and immunofluorescent imaging (FIG. 5B) of mouse red blood cells enzymatically ligated with B-TL5 or control mouse red blood cells revealed that conjugation was also successful in mouse red blood cells. As illustrated by the FACS histogram, over 99% of mouse red blood cells were successfully conjugated with the peptide. More importantly, this also indicates that the extended approach demonstrated above including streptavidin-mediated conjugation and copper-free click chemistry can also be applied to mouse red blood cells, making pre-clinical trials to test the efficacy of this approach much simpler. Analysis of immunofluorescent images showed a significant difference in Anti-biotin-PE fluorescence between peptide ligated and unligated mouse red blood cells (FIG. 5C). The Pearson's R value from co-localization analysis revealed a value of 0.81 for biotinylated peptide ligated mouse red blood cells, indicating slightly less efficient conjugation as compared to human red blood cells, possibly owing to slightly different membrane proteomic compositions in red blood cells between the two species.

Comparison of different peptide C-terminal motifs also revealed that mRBCs could be efficiently ligated with peptides displaying a diverse range of C-terminal sequences (FIG. 5D). However, the trend differed form that seen with hRBCs, potentially owing to the differences in RBC membrane protein composition between human and mouse RBCs. Comparison of peptide length however revealed a similar trend, with all peptides from 8 to 28 amino acids (1-5 EAAAK repeats) ligating efficiently (FIG. 5E). The best yield was seen with the shortest peptide (1 EAAAK repeat, 8 amino acids), with the ligation yield decreasing with length from 13 to 18 amino acids. Interestingly, the 28 amino acid peptide (5 EAAAK repeats) had a comparable yield to the 8 amino acid single EAAAK repeat peptide (FIG. 5E).

### Conjugation of Red Blood Cells is Efficient and Versatile.

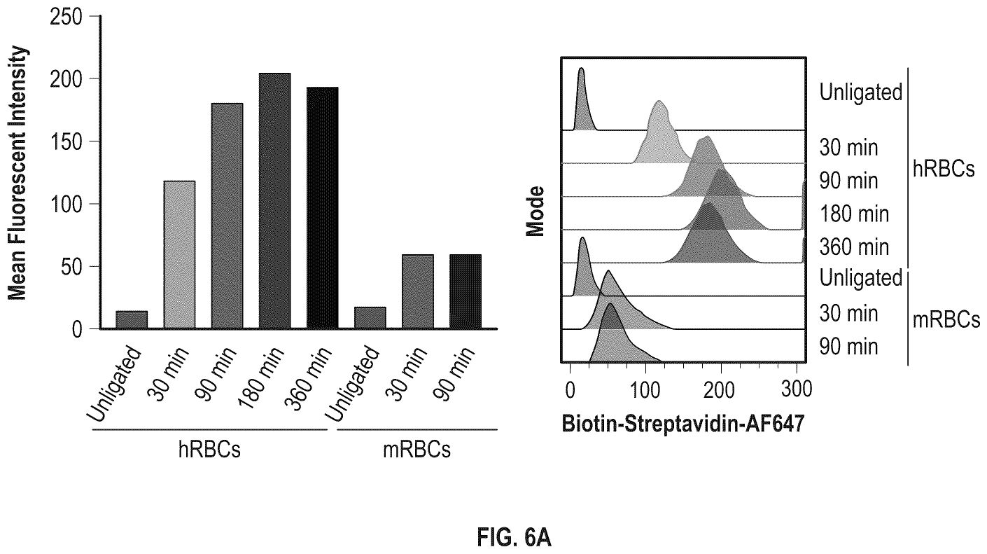

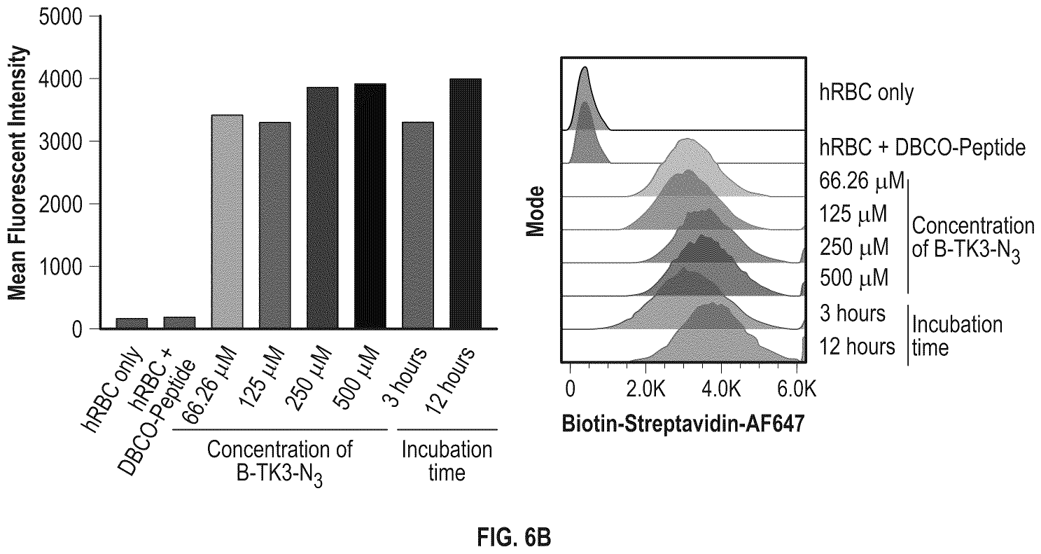

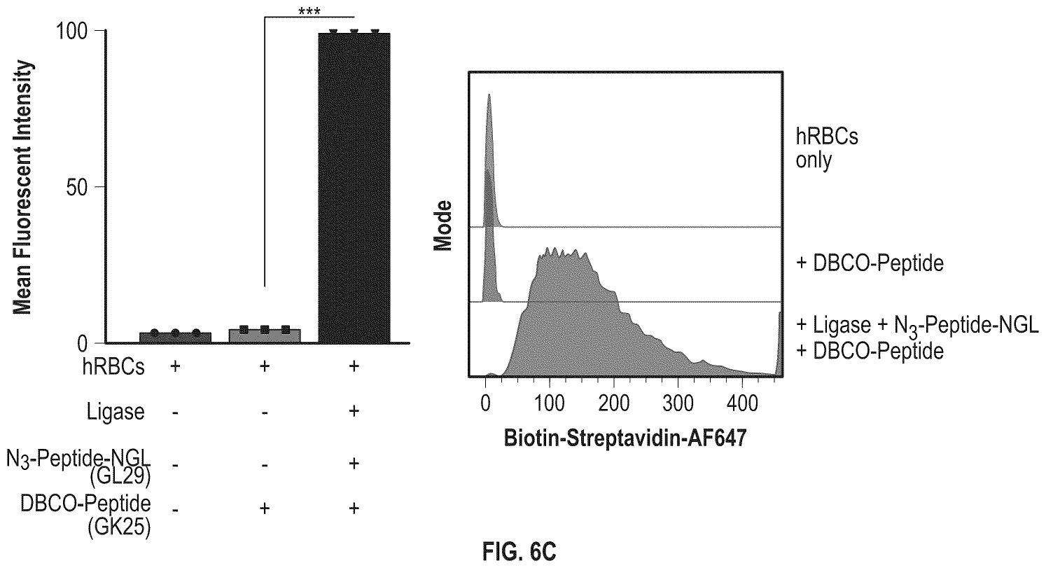

To ascertain the efficiency of enzymatic ligation, the time taken for complete conjugation of human and mouse red blood cells with B-TL5 peptide (FIG. 6A) was verified. Human red blood cell conjugation was saturated within 3 hours, with >90% efficiency achieved within 90 minutes. Mouse red blood cells were conjugated much faster (around 30 minutes), possibly due to the lower copy number of peptide on mouse red blood cells. Furthermore, extension of this approach via click chemistry was also assessed for efficiency and versatility. Successful click-chemistry mediated conjugation of 100% of human red blood cells was observed over a range of conditions, even with azido-peptide concentrations of 66 μM or incubation times as short at 3 hours (FIG. 6B). However, optimal conjugation was observed at about 250 μM peptide concentration, with an incubation time of up to 12 hours. FIG. 6C also demonstrates the possibility of conjugation with the orientation of functional groups switched (an azido-peptide ligated on the human red blood cells followed by incubation with a DBCO-tagged peptide), displaying the versatility of a combinatorial approach utilizing enzymatic ligation with copper-free click chemistry.

### The Ligation Protocol is Biocompatible and Results in Stable and Functional Conjugation In Vivo.

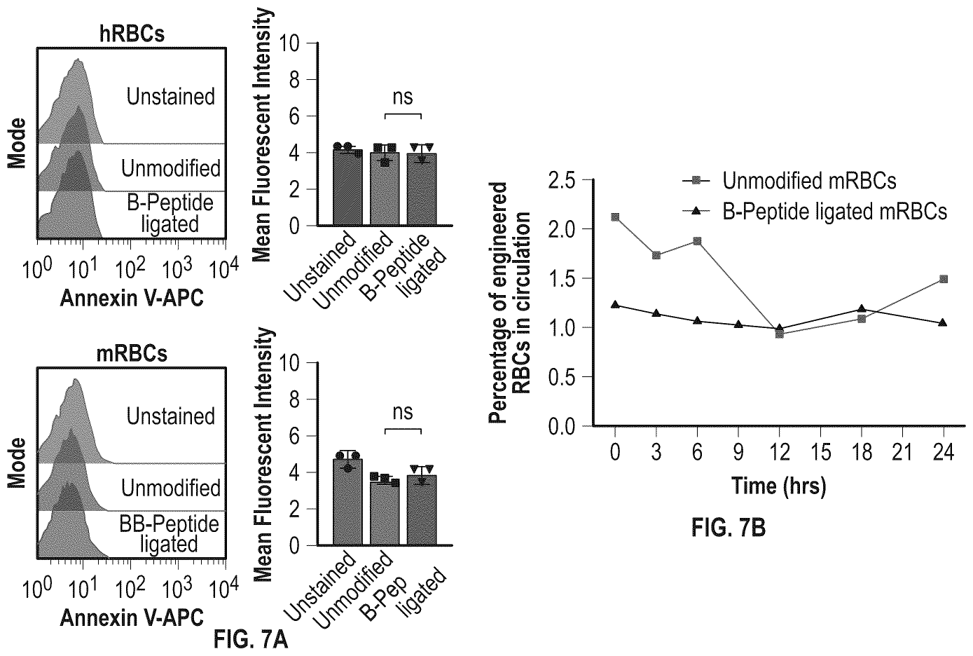

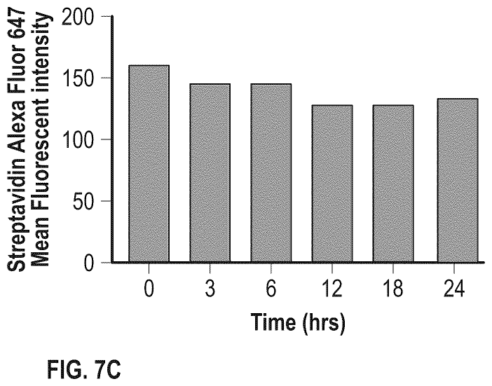

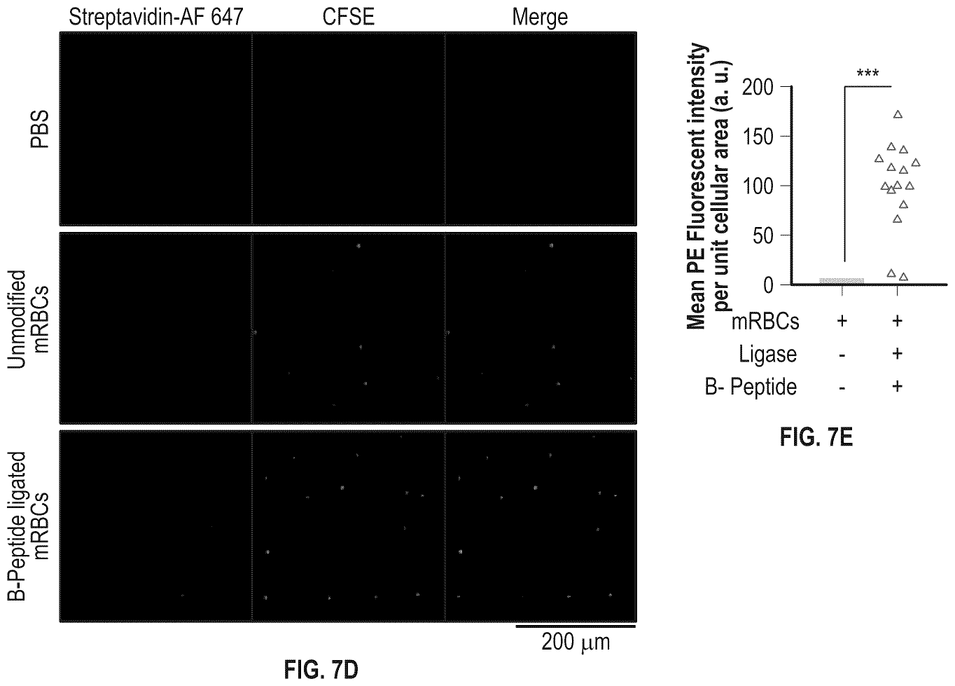

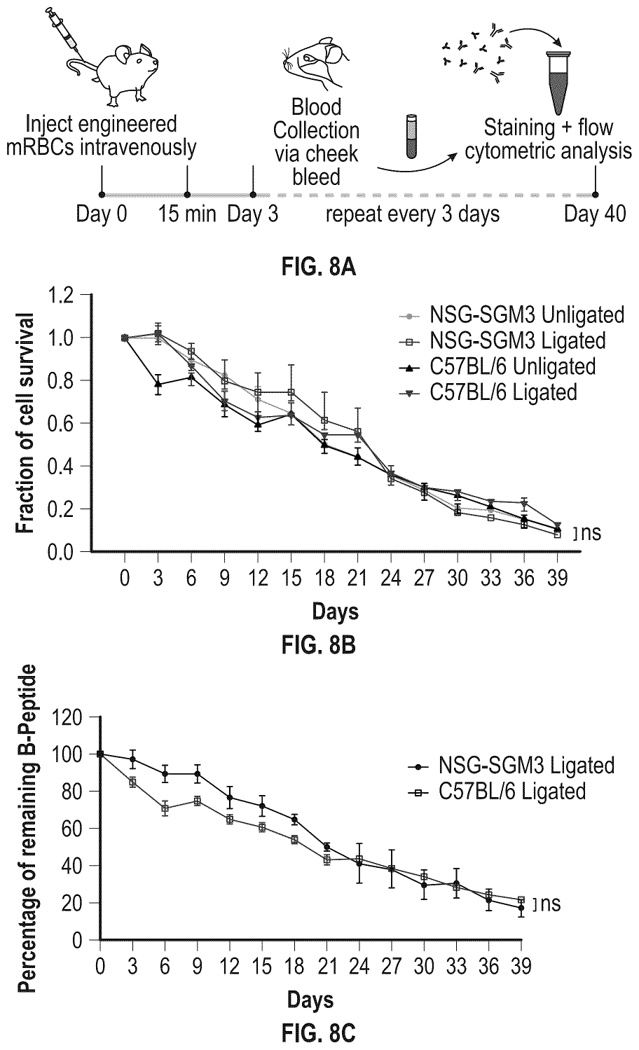

To test the biocompatibility of this method, biotinylated peptide ligated or unmodified human red blood cells and mouse red blood cells were analysed using Annexin V to test for the presence of PS which may signify induction of apoptosis. As shown in FIG. 7A, B-peptide ligated red blood cells showed no increase in Annexin V binding as compared to unmodified red blood cells, confirming the gentle and biocompatible nature of this method. Furthermore, we demonstrated the stability of engineered mouse red blood cells in vivo. To this end, B-TL5 peptide conjugated or control mouse red blood cells were labelled with CFSE and injected into mice via the tail vein. Blood was collected from the submandibular vein at regular interval for 24 hours. Cells were stained with Streptavidin Alexa Fluor 647 to detect biotin on the surface of mouse red blood cells. As illustrated by the scatter plot in FIG. 7B, engineered mouse red blood cells were retained in the blood as well as the unmodified (control) mouse red blood cells (detected using CFSE fluorescence). Moreover, the biotin tag on the N-terminal of the conjugated peptide remained stable for the duration of the experiment (FIG. 7C). FIG. 7D also shows representative immunofluorescent images of blood smears taken at 24 hours from mice injected with PBS or B-TL5 peptide ligated or unligated CFSE stained mouse red blood cells, confirming the stable nature of the engineered red blood cells in vivo. The same experiment was also carried out in immunodeficient NSG-SGM3 or immunocompetent C57BL/6 mice over a period of 39 days (FIG. 8A). As illustrated by the scatter plot in FIG. 8B, engineered mRBCs were retained in the blood as well as the unmodified (control) mRBCs (detected using CFSE fluorescence) in both mouse models. Remarkably, we could detect ˜50% of engineered RBCs remaining in circulation up to 21 days post-injection. Moreover, the biotin tag on the N-terminal of the conjugated peptide remained mostly stable for the duration of the experiment (FIG. 8C), though we do see a gradual decrease in signal, possible due to degradation of surface peptides after prolonged exposure to serum proteases. Despite this, we are still able to detect up to 50% of the original ligated product 3 weeks post-injection.

### Example 7: Comparison of Membrane Proteins in Red Blood Cells Compared to Red Blood Cell Derived Extracellular Vesicles

We have previously identified a list of candidate proteins on RBCEVs that were ligated with a biotinylated peptide by OaAEP1 ligase using a biotin pulldown experiment coupled with LFQ-Mass spectrometry (See FIG. 12) (Obtained from Pham et al, Journal of Extracellular Vesicles, 2021). Comparison of these candidate proteins, to a separate mass spectrometry of full RBCEV proteins revealed that 3 of the candidate proteins (ACKR1, ICAM4 and F11 R ranking 33^{rd}, 119^{th} and 57^{th} in relative abundance) are highly enriched in the RBCEVs compared to all other RBCEV proteins (relative abundance calculated based on LFQ (label-free quantitation) intensity.

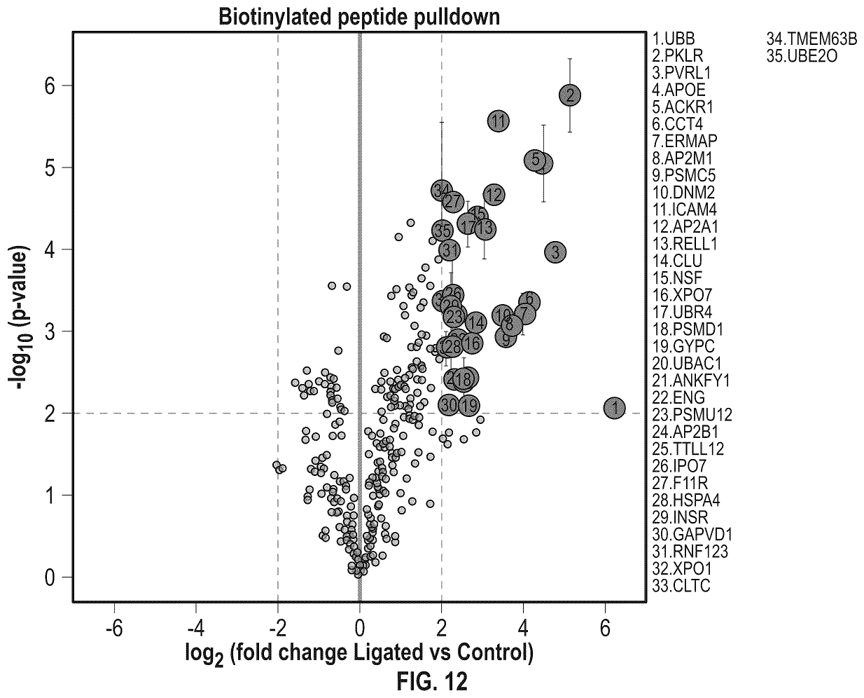

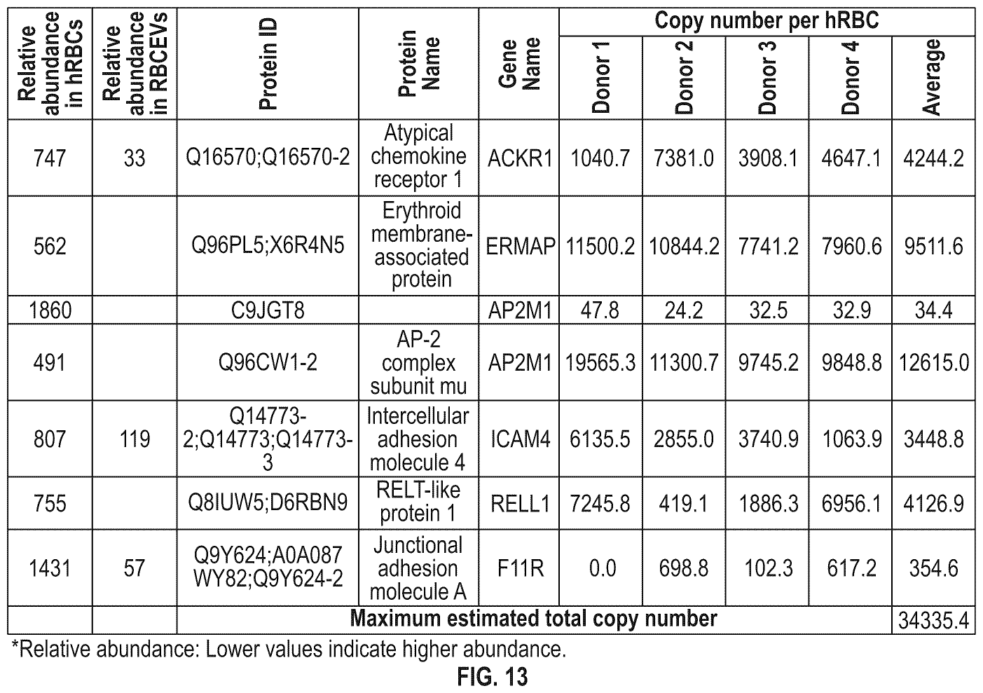

A cross-comparison of this data with existing RBC membrane proteome studies (see FIG. 13) demonstrates that in RBCs, these proteins are of very low relative abundance (all below 490) (Relative abundance obtained from ‘Quantitative analysis of human red blood cell proteome; Authors: Agata Bryk and Jacek R. Wisniewski (Biochemical Proteomics Group, Department of Proteomics and Signal Transduction, Max-Planck-Institute of Biochemistry, Am Klopferspitz 18, 82152 Martinsried, Germany)). Moreover, comparing their copy number demonstrates that if the same proteins as RBCEVs were ligated, it would result in very low copy numbers of peptides on RBCs, suggesting that the ligation approach may not be efficiently transferrable to RBCs.

This is in contrast to our ligation data that shows copies well in excess of 100,000. This indicates that different proteins are ligated in RBCs and RBCEVs. This is most likely due to changes in the membrane protein composition during vesiculation, either due to flipping of membranes dues to flippases and translocases or other modifications such as glycosylation that blocks ligation.

### Example 7: Comparison of Ligation Efficiency of Sortase and OaAEP1 Ligase

We compared the ligation efficiency of Sortase and OaAEP1 ligase. We have found that Sortase is unable to catalyse the ligation of single domain antibodies, either in a single step method (including on deglycosylated RBCs), or using a 2-step ligation using Sortase in both ligation steps—both to ligate the linker to the RBC, and to ligate the antibody to the linker. These methods resulted on essentially 0% ligation, in contrast to the striking efficiencies we have achieved with OaAEP1 ligase above.

To investigate this further, we investigated the ability of Sortase to ligate peptides with a suitable motif to human RBCs.

The following peptides were incubated with hRBCs overnight at room temperature:

| | |
|---|---|
| B-GG8 | Biotin-GLPETGGG |
| B-EK15-LPETGG | Biotin-EAAAKEAAAKEAAAKLPETGG |
| B-EK15-LPETGGG | Biotin-EAAAKEAAAKEAAAKLPETGGG |
| B-EK15-NGL | Biotin-EAAAKEAAAKEAAAKNGL |
| B-EK15-NDL | Biotin-EAAAKEAAAKEAAAKNDL |

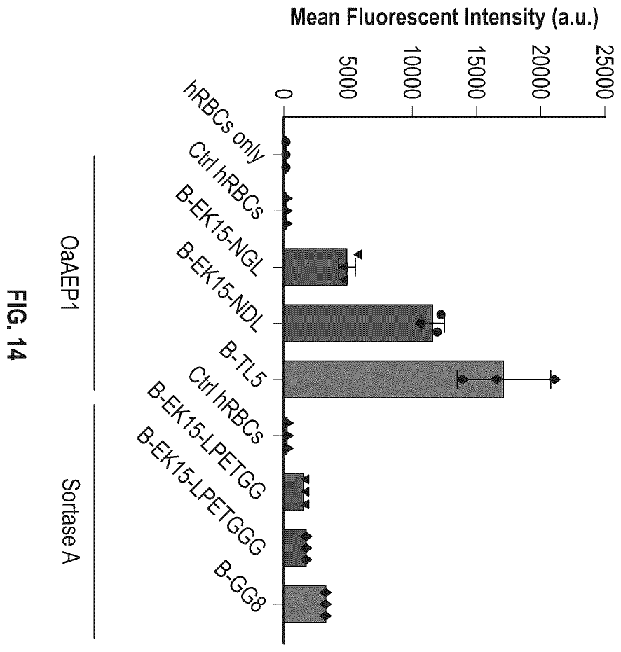

As shown in FIG. 14, Sortase is able to ligate suitable peptides to hRBCs, but does so at much lower yield as compared to OaAEP1 ligase. When comparing -LPETGG or -LPETGGG (the optimal Sortase A C-terminal motif) with -NGL or -NDL (the optimal C-terminal OaAEP1 motif), the ligation yield of Sortase is 5 and 10 times lower respectively.

### REFERENCES

A number of publications are cited above in order to more fully describe and disclose the invention and the state of the art to which the invention pertains. Full citations for these references are provided below. The entirety of each of these references is incorporated herein.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method comprising:
(a) contacting a Red Blood Cell (RBC) with a peptide in the presence of a ligase, under suitable conditions and for sufficient time to allow ligation of the peptide to the RBC to form an RBC-peptide conjugate; wherein the peptide comprises a C-terminal ligase recognition sequence; and optionally,
wherein the method further comprises a step of washing the RBC-peptide conjugate.

2. The method of claim 1 wherein the ligase is OaAEP1 ligase, and wherein the peptide is an effector molecule or a linker peptide.

3. (canceled)

4. The method of claim 2 wherein the peptide is an effector molecule, and the effector molecule has a C-terminal ligase recognition sequence selected from NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

5. (canceled)

6. The method of claim 2 wherein the peptide is a linker peptide and the linker peptide comprises a C-terminal ligase recognition sequence and a motif for conjugation to an effector molecule, where the motif for conjugation to another molecule is an N-terminal ligase recognition sequence, a click chemistry functional group, or a biotin moiety.

7. The method of claim 6 wherein the RBC-peptide conjugate is a RBC-linker peptide conjugate, and wherein the method further comprises:
(b) contacting the RBC-linker peptide conjugate with an effector molecule under suitable conditions and for sufficient time for conjugation of the effector molecule to the RBC-linker peptide to form an RBC-linker-effector molecule conjugate.

8. The method of claim 7 wherein the linker peptide comprises an N-terminal ligase recognition sequence and wherein the C-terminal ligase recognition sequence is Xaa₁GG, wherein Xaa₁ is any amino acid except G or has the sequence NG or NCL.

9. The method of claim 8 wherein the N-terminal ligase recognition sequence is G, GG, GL, GGG, GLG or GGL.

10. The method of claim 8 wherein the effector molecule comprises a C-terminal ligase recognition sequence selected from the group consisting of NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL; and/or wherein the RBC-linker peptide conjugate is contacted with the effector molecule under suitable conditions and for sufficient time for ligation of the effector molecule to the RBC-linker peptide to form an RBC-linker-effector molecule conjugate; and/or wherein the ligase in (a) is the same as the ligase in (b).

11-12. (canceled)

13. The method of claim 6 wherein the linker comprises a click chemistry functional group and a C-terminal ligase recognition sequence selected from the group consisting NGL, NDL, NPL, NAL, NCL, NEL, NFL, NHL, NKL, NIL, NLL, NQL, NRL, NSL, NTL, NVL, NWL, NYL, NSLD, NSLAN or NG, preferably NGL, NPL or NDL.

14. The method of claim 13 wherein the click chemistry functional group comprises azide moiety, a tetrazine moiety, a methyl tetrazine moiety a diarylcytooctyne (DBCO) moiety or a Transcyclooctyne (TCO) moiety.

15. The method of claim 14 wherein the effector molecule comprises a complementary click chemistry functional group.

16. The method of claim 13 wherein the RBC-linker conjugate is contacted with the effector molecule under suitable conditions and for sufficient time for conjugation of the effector molecule to the RBC-linker conjugate by Copper-free click chemistry.

17. The method of claim 6 wherein the RBC-peptide conjugate is a RBC-linker conjugate, wherein the linker comprises a biotin moiety, and wherein the method further comprises:
(b) contacting the RBC-linker peptide conjugate with streptavidin under suitable conditions and for sufficient time for conjugation of the biotin moiety of the RBC-linker peptide to streptavidin to form a RBC-linker-streptavidin conjugate; and
(c) contacting the RBC-linker-streptavidin conjugate with a biotinylated effector molecule under suitable conditions and for sufficient time for conjugation of the biotin moiety of the effector molecule to the streptavidin of the RBC-linker-streptavidin conjugate, thereby forming a RBC-linker-streptavidin-effector molecule conjugate.

18. (canceled)

19. The method of claim 2 wherein the peptide is a linker peptide, and the linker peptide comprises a linker body sequence, the linker body sequence:
(a) comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids;
(b) comprises an α-helical peptide sequence
(c) comprises repeats of the sequence EAAAK; or
(d) comprises the sequence EQKLISEEDL,
and/or wherein the peptide is a linker peptide, and the linker peptide consists of a sequence selected from the group consisting of:
GLGEQKLISEEDLGLPETGG;
DBCO-EAAAKEAAAKEAAAKNGL;
Azide-GSSGSGGEQKLISEEDLGGSGGSGSGNGL;
GLGEQKLISEEDLGLPETGG;
GGGEQKLISEEDLGLPETGG;
GLGEQKLISEEDLGNGL;
GGGEQKLISEEDLGNGL;
and
GLG(EAAAK)₅LPETGG.

20. (canceled)

21. The method according to claim 1 wherein the method RBC is deglycosylated.

22. A modified Red Blood Cell (RBC) produced by the method of claim 1.

23. A modified RBC comprising, on its exterior surface, a peptide, wherein the peptide is conjugated to a native red blood cell protein.

24. The modified RBC according to claim 23 wherein the peptide is an effector molecule or a linker protein.

25. (canceled)

26. The modified RBC according to claim 24 wherein the linker protein is further conjugated to an effector molecule.

27. The modified RBC according to claim 23 wherein the RBC is deglycoslyated.

28-31. (canceled)

32. The method of claim 2, wherein the OaAEP1 ligase is OaAEP1-Cys247Ala.

33. The method of claim 6, wherein the ligase is OaAEP1 ligase.

34. The method of claim 33, wherein the OaAEP1 ligase is OaAEP1-Cys247Ala ligase.
